SEQUENCE LISTING (111 sequences)

SEQ ID NO: 1 - protein, 20 aa, Mus musculus
INPANANSYYDKVLAHSRIR

SEQ ID NO: 2 - protein, 20 aa, Mus musculus
HSRIRGRDQGPNVCALQQIL

SEQ ID NO: 3 - protein, 20 aa, Mus musculus
LQQILGTKKKYFSSCKNWYQ

SEQ ID NO: 4 - protein, 20 aa, Mus musculus
KNWYQGAICGKKTTVLYECC

SEQ ID NO: 5 - protein, 20 aa, Mus musculus
LYECCPGYMRMEGMKGCPAV

SEQ ID NO: 6 - protein, 20 aa, Mus musculus
GCPAVMPIDHVYGTLGIVGA

SEQ ID NO: 7 - protein, 20 aa, Mus musculus
GIVGATTTQHYSDVSKLREE

SEQ ID NO: 8 - protein, 20 aa, Mus musculus
KLREEIEGKGSYTYFAPSNE

SEQ ID NO: 9 - protein, 20 aa, Mus musculus
APSNEAWENLDSDIRRGLEN

SEQ ID NO: 10 - protein, 20 aa, Mus musculus
RGLENNVNVELLNALHSHMV

SEQ ID NO: 11 - protein, 20 aa, Mus musculus
HSHMVNKRMLTKDLKHGMVI

SEQ ID NO: 12 - protein, 20 aa, Mus musculus
HGMVIPSMYNNLGLFINHYP

SEQ ID NO: 13 - protein, 20 aa, Mus musculus
INHYPNGVVTVNCARVIHGN

SEQ ID NO: 14 - protein, 20 aa, Mus musculus
VIHGNQIATNGVVHVIDRVL

SEQ ID NO: 15 - protein, 20 aa, Mus musculus
IDRVLTQIGTSIQDFLEAED

SEQ ID NO: 16 - protein, 20 aa, Mus musculus
LEAEDDLSSFRAAAITSDLL

SEQ ID NO: 17 - protein, 20 aa, Mus musculus
TSDLLESLGRDGHFTLFAPT

SEQ ID NO: 18 - protein, 20 aa, Mus musculus
LFAPTNEAFEKLPRGVLERI

SEQ ID NO: 19 - protein, 20 aa, Mus musculus
VLERIMGDKVASEALMKYHI

SEQ ID NO: 20 - protein, 20 aa, Mus musculus
MKYHILNTLQCSEAITGGAV

SEQ ID NO: 21 - protein, 20 aa, Mus musculus
TGGAVFETMEGNTIEIGCEG

SEQ ID NO: 22 - protein, 20 aa, Mus musculus
IGCEGDSISINGIKMVNKKD

SEQ ID NO: 23 - protein, 20 aa, Mus musculus
VNKKDIVTKNGVIHLIDEVL

SEQ ID NO: 24 - protein, 20 aa, Mus musculus
IDEVLIPDSAKQVIELAGKQ

SEQ ID NO: 25 - protein, 20 aa, Mus musculus
LAGKQQTTFTDLVAQLGLAS

SEQ ID NO: 26 - protein, 20 aa, Mus musculus
LGLASSLKPDGEYTLLAPVN

SEQ ID NO: 27 - protein, 20 aa, Mus musculus
LAPVNNAFSDDTLSMDQRLL

SEQ ID NO: 28 - protein, 20 aa, Mus musculus
DQRLLKLILQNHILKVKVGL

SEQ ID NO: 29 - protein, 20 aa, Mus musculus
VKVGLSDLYNGQILETIGGK

SEQ ID NO: 30 - protein, 20 aa, Mus musculus
TIGGKQLRVFVYRTAICIEN

SEQ ID NO: 31 - protein, 20 aa, Mus musculus
ICIENSCMVRGSKQGRNGAI

SEQ ID NO: 32 - protein, 20 aa, Mus musculus
RNGAIHIFREIIQPAEKSLH

SEQ ID NO: 33 - protein, 20 aa, Mus musculus
EKSLHDKLRQDKRFSIFLSL

SEQ ID NO: 34 - protein, 20 aa, Mus musculus
IFLSLLEAADLKDLLTQPGD

SEQ ID NO: 35 - protein, 20 aa, Mus musculus
TQPGDWTLFAPTNDAFKGMT

SEQ ID NO: 36 - protein, 20 aa, Mus musculus
FKGMTSEERELLIGDKNALQ

SEQ ID NO: 37 - protein, 20 aa, Mus musculus
KNALQNIILYHLTPGVYIGK

SEQ ID NO: 38 - protein, 20 aa, Mus musculus
VYIGKGFEPGVTNILKTTQG

SEQ ID NO: 39 - protein, 20 aa, Mus musculus
KTTQGSKIYLKGVNETLLVN

SEQ ID NO: 40 - protein, 20 aa, Mus musculus
TLLVNELKSKESDIMTTNGV

SEQ ID NO: 41 - protein, 20 aa, Mus musculus
TTNGVIHVVDKLLYPADIPV

SEQ ID NO: 42 - protein, 20 aa, Mus musculus
ADIPVGNDQLLELLNKLIKY

SEQ ID NO: 43 - protein, 20 aa, Mus musculus
KLIKYIQIKFVRGSTFKEIP

SEQ ID NO: 44 - protein, 20 aa, Mus musculus
FKEIPMTVYTTKIITKVVEP

SEQ ID NO: 45 - protein, 20 aa, Mus musculus
KVVEPKIKVIQGSLQPIIKT

SEQ ID NO: 46 - protein, 20 aa, Mus musculus
PIIKTEGPAMTKIQIEGDPD

SEQ ID NO: 47 - protein, 20 aa, Mus musculus
EGDPDFRLIKEGETVTEVIH

SEQ ID NO: 48 - protein, 20 aa, Mus musculus
TEVIHGEPVIKKYTKIIDGV

SEQ ID NO: 49 - protein, 20 aa, Mus musculus
IIDGVPVEITEKQTREERII

SEQ ID NO: 50 - protein, 20 aa, Mus musculus
EERIITGPEIKYTRISTGGG

SEQ ID NO: 51 - protein, 20 aa, Mus musculus
STGGGETGETLQKFLQKEVS

SEQ ID NO: 52 - protein, 20 aa, Mus musculus
QKEVSKVTKFIEGGDGHLFE

SEQ ID NO: 53 - protein, 20 aa, Mus musculus
GHLFEDEEIKRLLQGDTPAK

SEQ ID NO: 54 - protein, 20 aa, Mus musculus
DTPAKKIPANKRVQGPRRRS

SEQ ID NO: 55 - protein, 20 aa, Mus musculus
IPANKRVQGPRRRSREGRSQ

SEQ ID NO: 56 - protein, 20 aa, Homo sapiens
MIPFLPMFSLLLLLIVNPIN

SEQ ID NO: 57 - protein, 20 aa, Homo sapiens
VNPINANNHYDKILAHSRIR

SEQ ID NO: 58 - protein, 20 aa, Homo sapiens
HSRIRGRDQGPNVCALQQIL

SEQ ID NO: 59 - protein, 20 aa, Homo sapiens
LQQILGTKKKYFSTCKNWYK

SEQ ID NO: 60 - protein, 20 aa, Homo sapiens
KNWYKKSICGQKTTVLYECC

SEQ ID NO: 61 - protein, 20 aa, Homo sapiens
LYECCPGYMRMEGMKGCPAV

SEQ ID NO: 62 - protein, 20 aa, Homo sapiens
GCPAVLPIDHVYGTLGIVGA

SEQ ID NO: 63 - protein, 20 aa, Homo sapiens
GIVGATTTQRYSDASKLREE

SEQ ID NO: 64 - protein, 20 aa, Homo sapiens
KLREEIEGKGSFTYFAPSNE

SEQ ID NO: 65 - protein, 20 aa, Homo sapiens
APSNEAWDNLDSDIRRGLES

SEQ ID NO: 66 - protein, 20 aa, Homo sapiens
RGLESNVNVELLNALHSHMI

SEQ ID NO: 67 - protein, 20 aa, Homo sapiens
HSHMINKRMLTKDLKNGMII

SEQ ID NO: 68 - protein, 20 aa, Homo sapiens
NGMIIPSMYNNLGLFINHYP

SEQ ID NO: 69 - protein, 20 aa, Homo sapiens
INHYPNGVVTVNCARIIHGN

SEQ ID NO: 70 - protein, 20 aa, Homo sapiens
IIHGNQIATNGVVHVIDRVL

SEQ ID NO: 71 - protein, 20 aa, Homo sapiens
IDRVLTQIGTSIQDFIEAED

SEQ ID NO: 72 - protein, 20 aa, Homo sapiens
IEAEDDLSSFRAAAITSDIL

SEQ ID NO: 73 - protein, 20 aa, Homo sapiens
TSDILEALGRDGHFTLFAPT

SEQ ID NO: 74 - protein, 20 aa, Homo sapiens
LFAPTNEAFEKLPRGVLERI

SEQ ID NO: 75 - protein, 20 aa, Homo sapiens
VLERIMGDKVASEALMKYHI

SEQ ID NO: 76 - protein, 20 aa, Homo sapiens
MKYHILNTLQCSESIMGGAV

SEQ ID NO: 77 - protein, 20 aa, Homo sapiens
MGGAVFETLEGNTIEIGCDG

SEQ ID NO: 78 - protein, 20 aa, Homo sapiens
IGCDGDSITVNGIKMVNKKD

SEQ ID NO: 79 - protein, 20 aa, Homo sapiens
VNKKDIVTNNGVIHLIDQVL

SEQ ID NO: 80 - protein, 20 aa, Homo sapiens
IDQVLIPDSAKQVIELAGKQ

SEQ ID NO: 81 - protein, 20 aa, Homo sapiens
LAGKQQTTFTDLVAQLGLAS

SEQ ID NO: 82 - protein, 20 aa, Homo sapiens
LGLASALRPDGEYTLLAPVN

SEQ ID NO: 83 - protein, 20 aa, Homo sapiens
LAPVNNAFSDDTLSMDQRLL

SEQ ID NO: 84 - protein, 20 aa, Homo sapiens
DQRLLKLILQNHILKVKVGL

SEQ ID NO: 85 - protein, 20 aa, Homo sapiens
VKVGLNELYNGQILETIGGK

SEQ ID NO: 86 - protein, 20 aa, Homo sapiens
TIGGKQLRVFVYRTAVCIEN

SEQ ID NO: 87 - protein, 20 aa, Homo sapiens
VCIENSCMEKGSKQGRNGAI

SEQ ID NO: 88 - protein, 20 aa, Homo sapiens
RNGAIHIFREIIKPAEKSLH

SEQ ID NO: 89 - protein, 20 aa, Homo sapiens
EKSLHEKLKQDKRFSTFLSL

SEQ ID NO: 90 - protein, 20 aa, Homo sapiens
TFLSLLEAADLKELLTQPGD

SEQ ID NO: 91 - protein, 20 aa, Homo sapiens
TQPGDWTLFVPTNDAFKGMT

SEQ ID NO: 92 - protein, 20 aa, Homo sapiens
FKGMTSEEKEILIRDKNALQ

SEQ ID NO: 93 - protein, 20 aa, Homo sapiens
KNALQNIILYHLTPGVFIGK

SEQ ID NO: 94 - protein, 20 aa, Homo sapiens
VFIGKGFEPGVTNILKTTQG

SEQ ID NO: 95 - protein, 20 aa, Homo sapiens
KTTQGSKIFLKEVNDTLLVN

SEQ ID NO: 96 - protein, 20 aa, Homo sapiens
TLLVNELKSKESDIMTTNGV

SEQ ID NO: 97 - protein, 20 aa, Homo sapiens
TTNGVIHVVDKLLYPADTPV

SEQ ID NO: 98 - protein, 20 aa, Homo sapiens
ADTPVGNDQLLEILNKLIKY

SEQ ID NO: 99 - protein, 20 aa, Homo sapiens
KLIKYIQIKFVRGSTFKEIP

SEQ ID NO: 100 - protein, 20 aa, Homo sapiens
FKEIPVTVYTTKIITKVVEP

SEQ ID NO: 101 - protein, 20 aa, Homo sapiens
KVVEPKIKVIEGSLQPIIKT

SEQ ID NO: 102 - protein, 20 aa, Homo sapiens
PIIKTEGPTLTKVKIEGEPE

SEQ ID NO: 103 - protein, 20 aa, Homo sapiens
EGEPEFRLIKEGETITEVIH

SEQ ID NO: 104 - protein, 20 aa, Homo sapiens
TEVIHGEPIIKKYTKIIDGV

SEQ ID NO: 105 - protein, 20 aa, Homo sapiens
IIDGVPVEITEKETREERII

SEQ ID NO: 106 - protein, 20 aa, Homo sapiens
EERIITGPEIKYTRISTGGG

SEQ ID NO: 107 - protein, 20 aa, Homo sapiens
STGGGETEETLKKLLQEEVT

SEQ ID NO: 108 - protein, 20 aa, Homo sapiens
QEEVTKVTKFIEGGDGHLFE

SEQ ID NO: 109 - protein, 20 aa, Homo sapiens
GHLFEDEEIKRLLQGDTPVR

SEQ ID NO: 110 - protein, 20 aa, Homo sapiens
DTPVRKLQANKKVQGSRRRL

SEQ ID NO: 111 - protein, 20 aa, Homo sapiens
LQANKKVQGSRRRLREGRSQ

(19) United States
(12) Patent Application Publication
(10) Pub. No.: US 2011/0033516 A1
(43) Pub. Date: Feb. 10, 2011

(54) METHODS AND COMPOSITIONS FOR BONE HEALING BY PERIOSTIN

(75) Inventors: Roger R. Markwald, Mount Pleasant, SC (US); Michael J. Kern, Mount Pleasant, SC (US); Russell A. Norris, Charleston, SC (US); Kyle P. Kokko, Charleston, SC (US)

Correspondence Address: MYERS BIGEL SIBLEY & SAJOVEC, PO BOX 37428, RALEIGH, NC, 27627, US

(73) Assignee: Medical University of South Carolina

(21) Appl. No.: 12/851,100

(22) Filed: Aug. 5, 2010

Related U.S. Application Data
(60) Provisional application No. 61/231,742, filed on Aug. 6, 2009.

(51) Int. Cl. A61K 38/16; A61K 9/00; A61F 2/28; A61P 19/00

(52) U.S. Cl. 424/422; 514/16.7; 424/484

(57) ABSTRACT

The present invention provides methods and compositions for increasing bone production and/or decreasing bone fracture healing time in a subject, by administering an effective amount of periostin and/or active peptides and/or fragments thereof.

### STATEMENT OF PRIORITY

The present application claims the benefit, under 35 U.S.C. §119(e), of U.S. Provisional Application No. 61/231,742, filed Aug. 6, 2009, the entire contents of which are incorporated by reference herein.

### BACKGROUND OF THE INVENTION

Nearly 37 million musculoskeletal injuries occur annually in the U.S. and account for $69 billion, or 12% of total medical spending. Trauma related orthopedic conditions account for 1.9 million hospitalizations and require over $30 billion in total charges annually. In addition, patients experience pain, loss of function and temporary and/or total disability from skeletal trauma and fracture nonunions. Regardless of the type of bone fracture (e.g., simple, comminuted, open), it is clear that the development of novel, affordable therapeutics is essential to promote and enhance the patient's healing and quality of life. Although healing time is age related, with younger patients healing faster, bone regeneration still takes at least 6 weeks to heal, with 90% of patients being completely healed after 3-6 months. However, 5-10% of these patients fail to heal at all, with fracture nonunions being the most prevalent cause.

A fracture nonunion is defined as a fracture that is nine months old with failure to show signs of healing for three consecutive months¹. In patients with severe trauma resulting in an open fracture with segmental bone loss, nonunion is more common. There are many instances where despite optimal surgical intervention, seemingly benign fractures proceed to nonunion. In both of these cases, the structural, mechanical and biological processes are unable to adapt to the altered microenvironment of the bony injury. As a result, most of these nonunion cases require additional surgical intervention.

Nonunions are more likely to occur when fractures are open, infected, have segmental bone loss, have impaired blood supply, are comminuted or are distracted. Furthermore, nonunions often occur when there is failure of fixation, early mobilization, ill-advised open reduction, and when there is a fracture of previously irradiated bone¹⁰. It is important to accurately diagnose the types of nonunions (e.g., hypertrophic, oligotrophic, atrophic, infected and synovial pseudoarthrosis), so that appropriate treatment can be provided. Ultimately, the goals of treating nonunions are the same as treating acute fractures. That is, to promote mechanical stability with bone-to-bone contact at the fracture site while maintaining adequate bone vascularity. Meeting these goals can prove to be difficult in the severely injured patient (e.g., open fracture, segmental bone loss, severe soft tissue damage) or with poor planning, poor implementation or a combination thereof¹¹.

There are many methods to treat nonunions and they can be divided into three categories as follows: mechanical methods (e.g., plates, screws, nails); biologic methods (e.g., bone grafts, graft substitutes, growth factors); and a combination of mechanical and biologic methods. Recently, the development of biologic mediators has become an area of significant clinical interest. The American Academy of Orthopaedic Surgeons (AAOS), in cooperation with the Orthopaedic Trauma Association (OTA), cites the development of biologic mediators and delivery systems for molecular compounds for the treatment of nonunions and the acceleration of normal healing following acute trauma as the primary aims for orthopedic basic, clinical, and translational research for the treatment of major limb trauma^{12,13}. Four clinical situations in which the application of therapeutic agents should be considered include simple closed fractures, fracture nonunions, delayed fracture unions and fractures with segmental bone loss.

Current biological therapeutics have primarily focused on the use of bone morphogenic proteins (BMPs) to potentiate bone repair². BMPs are the most researched and best characterized of all the biologic therapeutics. The BMPs are a subfamily of the TGF-β superfamily of polypeptides that bind to cell surface receptors and initiate a myriad of intracellular cascades required for bone repair. BMPs have been shown to induce chemotaxis, migration, proliferation, and differentiation of mesenchymal stem cells¹⁴. Exogenously administered rhBMP-2 and rhBMP-7 (also named OP-1) have been extensively evaluated in animal models and human studies.

Periostin, the product of a BMP responsive gene³, is a secreted, 90 kDa matricellular protein, which specifically interacts with components of the extracellular milieu (e.g., collagen type I, fibronectin, tenascin-C) and members of the integrin family at the cell membrane⁴⁻⁷. Through these interactions, periostin functions as a mechanosensor relaying changes in the external environment to the integrins which, in turn, activate a host of signaling pathways resulting in the activation or repression of gene programs. At the cellular level, these molecular changes ultimately regulate cell processes such as migration, differentiation, proliferation and apoptosis. As its name implies, periostin is intensely expressed in the adult periosteum^{8,9}.

Periostin is evolutionarily conserved from mammals to bacteria and contains four repeated domains related to the Drosophila midline fascilin-1 gene (FIG. 1). The mammalian fascilin gene family comprises four members: periostin, βIG-H3, stabilin-1 and stabilin-2, all of which have been demonstrated to play important roles in cellular processes such as adhesion, migration and differentiation. Periostin was originally described as being specifically expressed by osteoblasts in vitro (MC3T3-L1 cell line) and in the periosteum and periodontal ligament in vivo^{15,16}. It was shown that periostin is regulated by the BMP responsive transcription regulator, Twist1, and is important for intramembranous ossification¹⁷. Periostin has been shown to specifically bind to collagen type I, promote collagen cross-linking and ultimately affect the biomechanical properties of connective tissues⁴.

The present invention provides methods and compositions comprising a periostin protein and active peptides and fragments thereof for treatment of bone fractures and for accelerating healing of bone fractures.

### SUMMARY OF THE INVENTION

The present invention provides, in one aspect, a method of increasing bone production in a subject (e.g., a subject in need thereof), comprising administering to the subject an effective amount of a periostin protein or a biologically active fragment thereof and/or a peptide of this invention and/or an effective amount of a nucleic acid or virus particle of this invention and/or an effective amount of a composition of this invention as described herein.

In addition, the present invention provides a method of decreasing healing time of a bone fracture in a subject (e.g., a subject in need thereof), comprising administering to the subject an effective amount of a periostin protein or a biologically active fragment thereof and/or a peptide of this invention and/or an effective amount of a nucleic acid and/or virus particle of this invention and/or an effective amount of a composition of this invention as described herein.

Further provided is a method of stimulating and/or accelerating ligament and tendon healing in a subject (e.g., a subject in need thereof), comprising administering to the subject an effective amount of a periostin protein or a biologically active fragment thereof and/or a peptide of this invention and/or an effective amount of a nucleic acid and/or virus particle of this invention and/or an effective amount of a composition of this invention as described herein.

In some embodiments, the methods of this invention can further comprise, consist of or consist essentially of administering to the subject an agent selected from the group consisting of: a) collagen; b) a hydrogel (either natural or synthetic); c) a demineralized bone matrix; d) an organic sponge; e) an implantable matrix; f) a bone chip (either allograft or autograft); and g) any combination of (a)-(f) above.

In additional embodiments, the methods of this invention can further comprise, consist of or consist essentially of administering to the subject an agent selected from the group consisting of: a) a differentiation stimulating agent; b) a chemotaxis stimulating agent; c) a proliferation stimulating agent; d) a mobilization stimulating agent; and e) any combination of (a)-(d) above.

In yet further embodiments, the methods of this invention can also comprise, consist essentially of or consist of administering to the subject an agent selected from the group consisting of: a) collagen; b) a hydrogel (either natural or synthetic); c) a demineralized bone matrix; d) an organic sponge; e) an implantable matrix; f) a bone chip (either allograft or autograft); g) a differentiation stimulating agent; h) a chemotaxis stimulating agent; i) a proliferation stimulating agent; j) a mobilization stimulating agent; and k) any combination of (a)-(j) above.

The present invention additionally provides an isolated peptide comprising, consisting essentially of and/or consisting of the amino acid sequence as set forth in any of SEQ ID NOs:1-55 and any combination thereof. The present invention additionally provides an isolated peptide or fragment of a human periostin protein as set forth in SEQ ID NOs:56-111, as well as an isolated peptide or fragment of a human periostin protein that is substantially similar to and/or equivalent in activity to a peptide having an amino acid sequence as set forth in SEQ ID NOs:1-55 and any combination thereof (i.e., a peptide having an amino acid sequence of a human periostin protein as set forth under the GenBank® Accession numbers provided herein and as are well known in the art). Further provided is a composition comprising the isolated peptide or combination thereof of this invention, with or without a full length periostin protein or biologically active fragment thereof, in a pharmaceutically acceptable carrier.

In further aspects, the present invention provides an isolated nucleic acid and/or a virus particle comprising a nucleotide sequence encoding a periostin protein or biologically active fragment thereof and/or an isolated peptide or combination thereof of this invention, which nucleic acid and/or virus particle can be present in a pharmaceutically acceptable carrier.

The compositions of this invention as described above can further comprise an agent, which can be but is not limited to: a) collagen; b) a hydrogel (either natural or synthetic); c) a demineralized bone matrix; d) an organic sponge and/or implantable matrix; e) a bone chip (either allograft or autograft); and f) any combination of (a)-(e) above.

### BRIEF DESCRIPTION OF THE DRAWINGS

FIG. 1 Schematic of periostin domain architecture. Fasciclin domains (Fas1-4), signal sequence (S.S.), cysteine rich domain (Cys), heparin binding domain (grey ovals), putative glycosylation site, and stop codon (asterisk) are depicted.

FIGS. 2A-D Stages of fracture healing. (A) Following fracture, periosteal and endosteal proliferation and migration occur to bridge the fractured area. (B) Hyaline cartilage is laid down by pre-osteoblast cells, followed by differentiation of these cells into bone forming osteoblasts. (C) This results in the formation of primary bone and callus that eventually become remodeled into secondary bone. (D) Over time mature bone is regenerated, resulting in a healed fracture. Adapted from Junqueria and Carneiro; Basic Histology 10^{th} edition.

FIGS. 3A-B Periostin regulates collagen synthesis. Mesenchyme from periostin null mice was placed in hanging drop cultures and incubated with either purified periostin protein (10 μg/ml) or PBS. Mesenchyme from wild-type mice was used as control tissue. After 7 days, tissues were harvested and analyzed for periostin and collagen expression by immunoblotting. (A) Periostin null mesenchyme exhibits low levels of fibroblastic markers: collagen Ia1 and Ia2. Addition of periostin to the culture medium induces expression of collagen Ia1 and Ia2. Actin is used for protein normalization. (B) Graphical representation of Western analyses presented in panel A obtained from densitometric analyses using NIH image software. Values obtained were compared against wild-type values (baseline) and represented as relative percent change. * denotes p<0.05.

FIG. 4 Periostin co-localizes with collagen type 1 in the periosteum. Immunohistochemical analyses of periostin and collagen I expression in adult mouse fibula showing significant overlap of expression in the periosteum (p) whereas the bone marrow (bm) lacks any appreciable staining.

FIG. 5 Adult periostin null bones are weaker than wild-type. Materials Testing System (MTS) analyses were performed on age-matched femurs isolated from periostin and wild-type mice. The periostin null femurs withstood a maximum force of 15 Newtons (N) whereas the wild-type mice were able to withstand 18 N's indicating the adult periostin null bones are significantly weaker than those of wild-type mice.

FIGS. 6A-B In vitro fibular osteotomy culture system. (A) Fibula fractures are embedded within a collagen hydrogel. After two hours, mesenchymal cells are seen migrating away from the fracture area with a significant increase in cell number after 15 hours. (B) Wild-type (WT) fibular osteotomies move closer to each other after 20 days (compare asterisk), eventually fusing during the sixth week of culture. Periostin knock-out (KO) bones are smaller in diameter (arrow head) and fail to move closer together (double asterisks).

FIGS. 7A-C Periostin is upregulated following bone fracture. Immunohistochemical analyses of periostin expression in (A) normal adult murine fibulas, (B) 2 weeks, and (C) 4 weeks post-fracture. (A) Periostin expression is confined to the periosteum in a normal, uninjured fibula (arrow head). (B) Two weeks following fibula osteotomy, periostin expression significantly increases in the skeletal muscle, callus (Ca) and bone marrow (BM) surrounding the fracture site. (C) By four weeks, expression of periostin is nearly undetectable within the bone marrow but still intense in the remodeling bone. Hoescht blue stain is used as a nuclear stain.

FIGS. 8A-C Periostin KO mice have defects in bone regeneration. X-ray analyses of bone healing three weeks after fibula osteotomies. (A) Wild-type mice exhibit pronounced healing, callus formation, and re-fusion after the initial fibular insult. (B,C) Fibula osteotomies were performed in the periostin null background and analyzed by X-ray three weeks later. In the absence of periostin there is no apparent callus formed and no fibula fusion (C). When purified periostin protein, in a hydrogel delivery format, is placed at the break point in the periostin null mouse, pronounced healing is evident (B). This demonstrates that periostin promotes bone regeneration in vivo.

FIG. 9 Genetic deletion of periostin delays bone fracture healing. Fibula osteotomies were performed on the left leg for three age matched male mice of each genotype (periostin +/+ and −/−). The healing process was followed in each mouse by X-ray every week. Two representative mice are shown at 14 and 21 days post fracture. In every periostin −/− mouse, healing was dramatically delayed (arrow) as compared to age and gender matched mice that were periostin +/+ (arrowheads). Specifically, callus formation was evident within 14 days for +/+ mice whereas −/− mice did not exhibit visible callus throughout eight weeks.

FIG. 10 Periostin promotes osteoblast cell migration. Migration assay demonstrates that periostin promotes osteoblast migration. Tissue culture dishes were coated with either collagen, or collagen with titrating amounts of periostin. Cells were plated and allowed to adhere for 24 hours. A standard “wounding” or “scratch assay” was performed and measurements of cell migration into the scratch area were obtained. Data demonstrate that the combination of periostin plus collagen results in the most potent stimulation of migration. In addition, the amount of periostin can be titrated, further suggesting that osteoblast cells are sensitive and responsive to the amount of periostin produced.

FIG. 11 Periostin promotes migration of primary fibroblasts in 3D collagen gel assays. Hanging drop aggregates (50,000 cells/20 μl) were placed on top of collagen I hydrogels (1.5 mg/ml) and assayed for their ability to respond to exogenous factors (TGFβ3, BMP2, and periostin). Bar graphs of the fold-change in area of migration demonstrate that each of the proteins promoted migration of the fibroblasts, with periostin exhibiting the highest degree of migration. Pictures above the graph lines show representative images of the stimulated cultures with the cell migratory boundaries outlined.

FIG. 12 Schematic of 4 point mechanical testing of a mouse femur. The positions of each point are very specific in placement and the grey shaded areas are the regions analyzed using MicroCT.

FIG. 13 Series of X-rays. The control panel is from the contralateral leg and the rest of the panels are from 2, 3, and 4 weeks after surgery only on the operated leg. A well developed callus is apparent by 3-4 weeks.

FIG. 14 Image generated from MicroCT analysis of fibula osteotomies. The control panel is from the contralateral leg and the rest of the panels are from different mice at 2, 3, and 4 weeks after surgery. A well developed callus is apparent by 3-4 weeks.

FIG. 15 Schematic for periostin peptide synthesis. Black numbers indicate 54 sequential peptides. Grey numbers indicate peptides selected as described herein.

FIGS. 16A-B Cell adhesion assays. (A) Each of the 55 peptides (20 mers) of periostin was assayed in triplicate by a cell adhesion assay using the ROS cell line. Collagen I and poly-L-lysine were used as positive controls, whereas 1% BSA coated wells were negative controls for non-specific binding. A total of 7/55 peptides (2, 10, 11, 12, 22, 28, and 30) showed significant binding. (B) The MC3T3 cell line (pre-osteoblasts) was additionally tested using this approach and demonstrated a similar, albeit non-identical, pattern of peptide binding. These subtle differences may be attributed to different integrin profiles of the two cell types. These data demonstrate that a fragment of periostin contains binding activity of the full length protein.

FIGS. 17A-B Periostin carboxyl truncation mutants. (A) Schematic of periostin and design of truncation mutants. Each truncation mutant is generated with a FLAG epitope tag at the C-terminus. Fasciclin domains are indicated by Roman numerals. Within each fasciclin domain are two regions exhibiting high homology between periostin and other family members: grey boxes-YH domains; black boxes-H2 domains. Amino acid positions are depicted at the top. (B) Western analysis of each of the 10 truncation mutants and full length (811). (C) Empty vector transfected control.

### DETAILED DESCRIPTION OF THE INVENTION

The present invention is based on the unexpected discovery that periostin, as well as active fragments and/or peptides of periostin, promote and accelerate healing and repair of bone fractures, as well as healing and repair of ligament and tendon injury. Particular aspects of this invention are explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure that do not depart from the instant invention. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, nucleotide sequences, amino acid sequences and other references mentioned herein are incorporated by reference in their entirety.

### Embodiments of Compositions of this Invention

The present invention provides, in one aspect, an isolated peptide and/or fragment of a periostin protein that has activity in promoting bone development and/or accelerating healing of a bone fracture. The periostin peptides and/or fragments of this invention can also have activity in promoting and/or accelerating ligament and/or tendon healing. Thus, in particular embodiments, the present invention provides an isolated peptide or fragment comprising, consisting essentially of and/or consisting of the amino acid sequence as set forth in any of SEQ ID NOs:1-55, and any combination thereof. For example, the present invention can comprise, consist essentially of, and/or consist of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 of the peptides of Table 1 in any combination and/or ratio relative to one another and in any association with one another (e.g., as single peptides, as linked peptides or as a combination of both single and linked peptides, which can include any number and combination of peptides including repeats, linked in any order).

The peptides set forth in SEQ ID NO:1-55 are based on an 811 amino acid murine periostin protein (e.g., as provided as GenBank® Accession No. NP_{—}056599). The present invention further comprises, consists essentially of and/or consists of an equivalent or homologous peptide (e.g., 10 mer, 12 mer, 14 mer, 16 mer, 18 mer, 20 mer) and/or fragment of a human periostin protein (e.g., as provided in GenBank® Accession Nos. Q15063, NP_{—}001129408, NP_{—}001129407, NP_{—}001129406, NP_{—}006466, AA106710, AA106711, ABY86633, ABY86632, ABY86631, ABY86630, AAY154840, CAH70107, CAH70106, CAH70105, CAH70104, CAH73571 CAH73570, CAH73569, CAH73568, EAX08594, EAX08593, EAX08591, EAX08590, NP_{—}002205, BAH13247, BAH12690, BAG65419 and AAN17733, the entire contents of each of which are incorporated by reference herein). The peptides set forth in SEQ ID NO:56-111 are based on an 836 amino acid human periostin protein (e.g., as provided as GenBank® Accession No. Q15063).

The term “equivalent” in some embodiments of this invention means a human periostin peptide made up of or comprising amino acids that correspond to the same or similarly numbered amino acids in a murine periostin peptide or periostin peptide from a different non-human species or that the human periostin peptide has substantially similar identity or homology to the murine or other non-human periostin peptide (e.g., 60%, 70%, 75%, 80%, 85%, 90%, 95%, 97%, 98%, 99%, 100%). The term “equivalent” is also intended in some embodiments to mean a peptide of a human periostin protein having the same or similar biological activity or function as a peptide of a murine periostin protein or a peptide of a different non-human periostin protein. Such equivalent peptides would be readily produced and analyzed by one of ordinary skill in the art according to standard and well known methods, as well as according to the methods described herein.

Thus, in some embodiments, the present invention provides a peptide comprising, consisting essentially of and/or consisting of amino acids 1-20, 5-25, 10-30, 15-35, 20-40, 25-45, 30-50, 35-55, 40-60, 45-65, 50-70, 55-75, 60-80, 65-85, 70-90, 75-95, 80-100, 85-105, 90-110, 95-115, 100-120, 105-125, 110-130, 115-135, 120-140, 125-145, 130-150, 135-155, 140-160, 145-165, 150-170, 155-175, 160-180, 165-185, 170-190, 175-195, 180-200, 185-205, 190-210, 195-215, 200-220, 205-225, 210-230, 215-235, 220-240, 225-245, 230-250, 235-355, 240-260, 245-265, 250-270, 255-275, 260-280, 265-285, 270-290, 275-295, 280-300, 285-305, 290-310, 295-315, 300-320, 305-325, 310-330, 315-335, 320-340, 325-345, 330-350, 335-355, 340-360, 345-365, 350-370, 355-375, 360-380, 365-385, 370-390, 375-395, 380-400, 385-405, 390-410, 395-415, 400-420, 405-425, 410-430, 415-435, 420-440, 425-445, 430-450, 435-455, 440-460, 445-465, 450-470, 455-475, 460-480, 465-485, 470-490, 475-495, 480-500, 485-505, 490-510, 495-515, 500-520, 505-525, 510-530, 515-535, 520-540, 525-545, 530-550, 535-555, 540-560, 545-565, 550-570, 555-575, 560-580, 565-585, 570-590, 575-595, 580-600, 585-605, 590-610, 595-615, 600-620, 605-625, 610-630 615-635, 620-640, 625-645, 630-650, 635-655, 640-660, 645-665, 650-670, 655-675, 660-680, 665-685, 670-690, 675-695, 680-700, 685-705, 690-710, 695-715, 700-720, 705-725, 710-730, 715-735, 720-740, 725-745, 730-750, 735-755, 740-760, 745-765, 750-770, 755-775, 760-780, 765-785, 770-790, 775-795, 780-800, 785-805, 790-810, 795-815, 800-820, 805-825, 810-830, 815-835 and/or 820-835 singly or in any combination, of a periostin protein of this invention (.e.g., as set forth according to the numbering of amino acids in the amino acid sequence identified by the GenBank® Accession number Q15063, as set forth herein, all of these are incorporated by reference herein in their entireties, and as otherwise known in the art).

The present invention further provides a domain or fragment (e.g., a biologically active domain or fragment) of a periostin protein as described herein. Such a domain or fragment of this invention can comprise, consist essentially of and/or consist of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820 or 830 contiguous amino acids of the periostin protein of this invention (e.g., as set forth pursuant to the numbering of the amino acid sequence identified by the GenBank® Accession number Q15063, as set forth herein and as otherwise known in the art), starting from either the amino terminus, the carboxy terminus and/or any internal site and in any combination. Furthermore, the domain or fragment of this invention can be combined with any other domain or fragment, either in operable association therewith, as separate domains or fragments (e.g., in a composition) or both. As one nonlimiting example, a 30 amino acid fragment near the amino terminus of the periostin protein can be combined, either in operable association with or as part of a composition with, a different 20 amino acid fragment that may also be near the amino terminus or it may be near the carboxy terminus.

Further provided is a composition comprising, consisting essentially of and/or consisting of an isolated peptide or combination thereof of this invention, with or without a full length periostin protein and/or biologically active fragment thereof (e.g., a fragment of the periostin protein that has at least one activity of the full length periostin protein), in a pharmaceutically acceptable carrier. Also provided herein is a composition comprising, consisting essentially of and/or consisting of a fragment or domain of a periostin protein, with or without a full length periostin protein. Such compositions can further comprise any of the delivery components and/or biological agents of this invention, as described herein. In particular, the compositions of this invention can comprise other therapeutic agents (e.g., growth factors) that increase bone production and/or decrease healing time of a bone fracture and/or stimulate and/or accelerate ligament and tendon healing, as would be known to one of ordinary skill in the art.

In further aspects, the present invention provides an isolated nucleic acid and/or virus particle comprising a nucleotide sequence encoding a periostin protein and/or or biologically active fragment thereof and/or an isolated peptide or combination thereof of this invention, which virus particle can be present in a pharmaceutically acceptable carrier.

The compositions of this invention as described above can further comprise an agent (e.g., a delivery agent), which can be but is not limited to: a) a hydrogel (either natural or synthetic); b) a demineralized bone matrix; c) an organic sponge; d) a bone chip (either allograft or autograft); and e) any combination of (a)-(d) above.

### Embodiments of Methods of the Invention

The present invention is based on the discovery that periostin and/or active peptides and/or active fragments of periostin, as well as nucleic acids encoding any of these, can be administered to a subject to increase bone production and/or promote bone healing, including the healing and repair of bone fractures. It is expected that one or more of the peptides and/or fragments (in any combination) of this invention (e.g., as set forth in Table 1 and equivalents such as human equivalents as described herein) will bind to the receptor for periostin on or in a cell in which the intact periostin protein binds. Upon receptor binding, signaling of upregulation of bone producing cells including osteoblasts, mesenchymal cells and/or stem cells will occur, leading to increased bone production and decreased healing time.

Thus in one aspect, the present invention provides a method of increasing bone production in a subject (e.g., in a subject in need thereof), comprising administering to the subject an effective amount of a periostin protein and/or a biologically active fragment thereof and/or a peptide of this invention and/or an effective amount of a nucleic acid and/or virus particle of this invention and/or an effective amount of a composition of this invention as described herein.

In addition, the present invention provides a method of decreasing healing time of a bone fracture in a subject (e.g., in a subject in need thereof), comprising administering to the subject an effective amount of a periostin protein and/or a biologically active fragment thereof and/or a peptide of this invention and/or an effective amount of nucleic acid and/or virus particle of this invention and/or an effective amount of a composition of this invention as described herein.

Further embodiments of this invention include methods to accelerate ligament and tendon healing in a subject (e.g., a subject in need thereof), comprising administering to the subject an effective amount of a periostin protein and/or a biologically active fragment thereof and/or a peptide of this invention and/or an effective amount of nucleic acid and/or virus particle of this invention and/or an effective amount of a composition of this invention as described herein. Protocols to produce and analyze the effect of periostin protein and/or a biologically active fragment thereof and/or a peptide of this invention and/or a nucleic acid and/or a virus particle on ligament and/or tendon healing are the same as those used to produce and analyze the effect of these various materials on bone healing, as described herein and as would be well known to one of ordinary skill in the art.

It has been demonstrated that collagen deposition by fibroblasts is increased in a dose-dependent manner following periostin exposure/incubation. Upon injury of ligaments and tendons, a collagen matrix helps bridge the injured tissue and forms the scaffold for ligamentous and tendonous healing. Developing a new set of therapeutics to stimulate/accelerate tendon and ligament healing is of significant clinical and public health interest. Application of periostin and/or periostin peptides and/or fragments directly or indirectly to the site where tendon and/or ligament repair is desired or required can enhance the process of ligament and/or tendon repair, thereby improving speed of healing and quality of recovery.

Thus, further provided herein is a method of stimulating and/or accelerating ligament and tendon healing in a subject (e.g., a subject in need thereof), comprising administering to the subject an effective amount of a periostin protein or a biologically active fragment thereof and/or a peptide of this invention and/or an effective amount of a nucleic acid and/or virus particle of this invention and/or an effective amount of a composition of this invention as described herein.

In the methods of this invention, in some embodiments, a differentiation stimulating agent, a chemotaxis and/or proliferation stimulating agent and/or a mobilization stimulating agent, as well as nucleic acids encoding any of these can be administered to a subject of this invention, either before, after, and/or simultaneously with the administration of a periostin protein or a biologically active fragment thereof and/or a peptide of this invention and/or an effective amount of a nucleic acid and/or virus particle of this invention and/or an effective amount of a composition of this invention as described herein.

In some embodiments of the invention, the differentiation stimulating agent can be, but is not limited to, a bone morphogenic protein (BMP, including BMP-1, BMP-2, BMP-4, BMP-5, BMP-6, BMP-7, BMP-8a and/or BMP-9), a transforming growth factor (TGF), including TGF-alpha, TGF-beta 1, TGF-beta 2 and TGF-beta 3, vitamin B12, an insulin-like growth factor-I (e.g., IGF-I; Stem Cells 22:1152-1167 (2004)), IGF-II, or any combination thereof.

In other embodiments, the chemotaxis and/or proliferation stimulating agent can be, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), an interleukin (e.g., interleukin-8; interleukin-10), or any combination thereof.

In further embodiments of the invention, the mobilization stimulating agent can be, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), FGF, EGF, an interleukin (e.g., interleukin-8; interleukin-10) or any combination thereof.

In some embodiments of this invention, collagen and/or active fragments thereof can be administered to a subject of this invention, before, after and/or simultaneously with administration of the periostin protein or biologically active fragment thereof and/or peptide and/or nucleic acid and/or virus particle and/or composition of this invention

As noted above, in some embodiments of the methods of this invention, the periostin protein or biologically active fragment thereof and/or peptide and/or nucleic acid and/or virus particle and/or composition can be administered directly to an injury/trauma/wound/surgical site in the subject.

In further embodiments of the methods of this invention, the periostin protein or biologically active fragment thereof and/or peptide and/or virus particle and/or composition can be administered to the subject intravenously, intra-arterially, orally and/or transdermally.

In additional embodiments, the nucleic acid and/or virus particle of this invention can be introduced into bone marrow stem cells of the subject according to methods well known in the art.

In the methods of this invention, an effective amount of the periostin protein or biologically active fragment thereof or the peptide is in the range of about 1 microgram/ml to about 500 milligrams/ml, with the optimum dosage for a given subject being routinely determined according to methods standard in the art (see, e.g., Remington's Pharmaceutical Sciences, latest edition).

The methods of this invention can further comprise delivering an effective amount of an anti-inflammatory agent, an effective amount of a cytokine, or a combination thereof to the injury/trauma/surgical/wound site of the subject to reduce and/or prevent inflammation and damage to tissue surrounding the site. Nonlimiting examples of an anti-inflammatory agent of this invention include steroids and nonsteroid anti-inflammatory agents as are well known in the art. Nonlimiting examples of a cytokine of this invention include anti-inflammatory cytokines such as IL-10, IL-4, IL-11, IL1Ra, TGF-β, osteoprotegerin and any combination thereof. The anti-inflammatory agents and cytokines of this invention can be delivered to the subject as a protein or active fragment thereof and/or as a nucleic acid encoding the protein or active fragment thereof. The amino acid sequences and nucleic acid sequences of exemplary anti-inflammatory agents and cytokines of this invention, as well as active fragments thereof are well known in the art and would be readily available to those skilled in the art. The periostin protein, peptides and/or fragments and/or nucleic acids of this invention, as well as the anti-inflammatory agents and cytokines, either as proteins or nucleic acids, can be administered in any combination and in any order relative to one another and in any time frame relative to one another.

In further embodiments of this invention, it is contemplated that a nucleic acid of this invention can be delivered to a subject of this invention, wherein the nucleic acid encodes a periostin protein, a peptide and/or fragment of this invention, and an antagonist of a pro-inflammatory agent. In some embodiments, the nucleic acid can be under the control of a promoter and/or other regulatory element such that expression of the nucleic acid is induced by a pro-inflammatory agent to be expressed to produce the periostin protein, peptide and/or fragment and antagonist of the pro-inflammatory agent. Nonlimiting examples of antagonists of pro-inflammatory agents include antagonists of TNFα, CSF-1, IL-6, IL 12, IL17, IL1B, receptor activator of nuclear factor-kappa B (RANK), RANK ligand (RANKL) and combinations thereof.

The present invention also provides various compositions. In some embodiments these compositions can be employed, e.g., in the methods described herein. Thus, the present invention provides a composition comprising, consisting essentially of and/or consisting of a periostin protein, a peptide and/or active fragment thereof and/or a nucleic acid encoding a periostin protein, peptide and/or active fragment thereof, which can be, for example, in a pharmaceutically acceptable carrier. Such compositions of this invention can further comprise, consist essentially of and/or consist of an anti-inflammatory agent, a cytokine, an immune modulator, an antagonist of a pro-inflammatory agent or any combination thereof and/or a nucleic acid encoding an anti-inflammatory agent, a cytokine, an immune modulator, an antagonist of a pro-inflammatory agent, a differentiation-stimulating agent, a chemotaxis stimulating agent, a proliferation stimulating agent, a mobilization stimulating agent or any combination thereof.

It is further contemplated that the present invention provides a kit comprising, consisting essentially of and/or consisting of compositions of this invention. It would be well understood by one of ordinary skill in the art that the kit of this invention can comprise one or more containers and/or receptacles to hold the reagents (e.g., periostin proteins, peptides and/or active fragments thereof, nucleic acids, viral vectors, etc.) of the kit, along with appropriate buffers and/or diluents and/or other solutions and directions for using the kit, as would be well known in the art. Such kits can further comprise anti-inflammatory agents, antagonists of pro-inflammatory agents and/or other cytokines, as well as nucleic acids encoding the same, in any combination, as described herein and as are well known in the art.

The compositions and kits of the present invention can also include other medicinal agents, pharmaceutical agents, carriers and diluents, etc. Actual methods of preparing such dosage forms are known, or will be apparent, to those skilled in this art.

In the kits of this invention, the compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

### Further Definitions

The following terms are used in the description herein and the appended claims:

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention, as well as the term “periostin,” encompasses any peptide, polypeptide, protein, analog, isoform or derivative of periostin, the nucleic acid sequences and amino acid sequences of which are well known in the art. Isoforms of periostin are also well known in the art, as set forth in the amino acid sequences identified by the GenBank® accession numbers provided herein.

Exemplary peptides of this invention are listed in Tables 1 and 2 and described in the Examples section provided herein. The periostin peptide, polypeptide, protein, isoform, analog and/or derivative thereof used in the present invention may be present in any amount that is sufficient to elicit a beneficial and/or therapeutic effect and, where applicable, may be present either substantially in the form of one optically pure enantiomer or as a mixture, racemic or otherwise, of enantiomers. As will be appreciated by those skilled in the art, the actual amount of peptide, polypeptide, protein, analogs and/or derivatives thereof used in the compositions of this invention will depend on the potency of the selected compound in question. The peptides, polypeptides, proteins, analogs and/or derivatives described herein may be obtained through commercial resources or may be prepared according to methods known to one skill in the art.

As used herein, “nucleic acid,” “nucleotide sequence” and “polynucleotide” encompass both RNA and DNA, including cDNA, genomic DNA, mRNA, synthetic (e.g., chemically synthesized) DNA and chimeras of RNA and DNA [e.g., DNA-RNA hybrid sequences (including both naturally occurring and non-naturally occurring nucleotides)], but are typically either single or double stranded DNA or RNA sequences.

The term polynucleotide or nucleotide sequence refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome or environment of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by oligonucleotide synthesis, PCR or restriction endonuclease treatment), independent of other sequences. It also includes a recombinant DNA that is part of a hybrid nucleic acid encoding an additional polypeptide or peptide sequence.

The term “isolated” can refer to a nucleic acid, nucleotide sequence, polypeptide, peptide or fragment that is at least partially and in some embodiments substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is at least partially separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated ex vivo and then returned to the subject.

The term “nucleic acid fragment” will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 60%, 65%, 70%, 75%, 80%, 85%, 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of and/or consist of, oligonucleotides having a length of at least about 6, 7, 8, 9, 10, 11, 12, 13, 14, 15, 16, 17, 18, 19, 20, 21, 22, 23, 24, 25. 50, 75, 100, 150, 200, 250, 300, 350, 400, 450, 500, 750, 1000, 1500, 2000, 2500, 3000, 4000 or 5000 consecutive nucleotides of a nucleic acid or nucleotide sequence according to the invention.

Several methods known in the art may be used to produce a polynucleotide and/or vector according to this invention. A “vector” is any nucleic acid molecule for the cloning and/or amplification of nucleic acid as well as for the transfer of nucleic acid into a subject (e.g., into a cell of the subject). A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral nucleic acid molecules for introducing a nucleic acid into a cell in vitro, ex vivo, and/or in vivo.

A large number of vectors known in the art may be used to manipulate nucleic acids, incorporate response elements and promoters into genes, nucleotide sequences, coding sequences, etc. Such vectors include, for example, plasmids or modified viruses including, for example bacteriophages such as lambda derivatives, or plasmids such as pBR322 or pUC plasmid derivatives, or the Bluescript® vector. For example, the insertion of the nucleic acid fragments or segments that function as response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate the nucleic acid and produce the proteins encoded by the marker.

Vectors have been used in a wide variety of gene delivery applications in cells, as well as in living animal subjects. Viral vectors that can be used include but are not limited to retrovirus, lentivirus, adeno-associated virus, poxvirus, alphavirus, baculovirus, vaccinia virus, herpes virus, Epstein-Barr virus, and adenovirus vectors, as well as any combination thereof. Nonlimiting examples of non-viral vectors include plasmids, liposomes, electrically charged lipids (cytofectins), nucleic acid-protein complexes, and biopolymers, as well as any combination thereof. In addition to a nucleic acid of interest, a vector may also comprise one or more regulatory regions (e.g., promoters, enhancers, termination sequences, etc.), and/or selectable markers useful in selecting, measuring, and monitoring nucleic acid transfer results (delivery to specific tissues, duration of expression, etc.).

“Promoter” refers to a nucleic acid sequence capable of controlling the expression of a coding sequence or functional RNA. In general, a coding sequence is located 3′ to a promoter sequence. Promoters may be derived in their entirety from a native sequence, or be composed of different elements derived from different promoters found in nature, or even comprise synthetic nucleic acid segments. It is understood by those skilled in the art that different promoters may direct the expression of a nucleotide sequence in different tissues or cell types and/or at different stages of development and/or in response to different environmental or physiological conditions.

Promoters that cause a nucleotide sequence to be expressed in most cell types at most times are commonly referred to as “constitutive promoters.” Promoters that cause a nucleotide sequence to be expressed in a specific cell type are commonly referred to as “cell-specific promoters” or “tissue-specific promoters.” Promoters that cause a nucleotide sequence to be expressed at a specific stage of development or cell differentiation are commonly referred to as “developmentally-specific promoters” or “cell differentiation-specific promoters.” Promoters that are induced and cause a nucleotide sequence to be expressed following exposure or treatment of the cell with an agent, biological molecule, chemical, ligand, light, or the like that induces the promoter are commonly referred to as “inducible promoters” or “regulatable promoters.” It is further recognized that, because in most cases the exact boundaries of regulatory sequences have not been completely defined, nucleotide sequences of different lengths may have identical promoter activity.

A “promoter sequence” is a nucleic acid regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence can be found a transcription initiation site (defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are nucleic acid regulatory sequences, such as promoters, enhancers, terminators, and the like, that provide for the expression of a coding sequence in a cell. For example, in eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in sense and/or antisense orientation.

The nucleic acids or plasmids or vectors may further comprise at least one promoter suitable for driving expression of a nucleotide sequence in a cell. The term “expression vector” means a vector, plasmid or vehicle designed to enable the expression of an inserted nucleotide sequence following delivery of a nucleotide sequence into a cell. The cloned nucleotide sequence, i.e., the inserted nucleotide sequence, is usually placed under the control of control elements such as a promoter, a minimal promoter, an enhancer, or the like. Initiation control regions or promoters, which are useful to drive expression of a nucleic acid in a cell are numerous and familiar to those skilled in the art. Virtually any promoter capable of driving expression of a nucleotide sequence is suitable for the present invention, including but not limited to, viral promoters, bacterial promoters, animal promoters, mammalian promoters, synthetic promoters, constitutive promoters, tissue specific promoters, developmental specific promoters, inducible promoters, and/or light regulated promoters.

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, and/or a nucleic acid vector transporter in any order and in any combination (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In some embodiments, a polynucleotide or nucleic acid of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO 95/18863 and WO 96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as bone marrow, pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically.

In various embodiments, other molecules can be used for facilitating delivery of a nucleic acid in vivo, such as a cationic oligopeptide (e.g., as described in International Patent Publication No. WO 95/21931), peptides derived from nucleic acid binding proteins (e.g., as described in International Patent Publication No. WO 96/25508), and/or a cationic polymer (e.g., as described in International Patent Publication No. WO 95/21931).

It is also possible to deliver a nucleic acid of this invention to a subject in vivo as naked nucleic acid (see, e.g., U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” means the uptake of exogenous or heterologous nucleic acid (RNA and/or DNA) by a cell. An “exogenous nucleotide sequence,” “heterologous nucleotide sequence” or “exogenous or heterologous nucleic acid” is typically a nucleotide sequence or nucleic acid molecule that is not naturally occurring in the virus genome in which it is present and/or is not naturally occurring in the cell into which it is introduced or is not naturally occurring in the cell into which it is introduced in the form and/or amount in which it is present in the cell upon introduction. Generally, the heterologous nucleic acid or nucleotide sequence comprises an open reading frame that encodes a peptide, a polypeptide and/or a nontranslated functional RNA.

A cell has been “transfected” with an exogenous or heterologous nucleic acid when such nucleic acid has been introduced or delivered inside the cell. A cell has been “transformed” by exogenous or heterologous nucleic acid when the transfected nucleic acid imparts a phenotypic change in the cell and/or in an activity or function of the cell. The transforming nucleic acid can be integrated (covalently linked) into chromosomal DNA making up the genome of the cell and/or it can be present as a plasmid (e.g., stably integrated and/or transient).

As used herein, “transduction” of a cell means the transfer of genetic material into the cell by the incorporation of nucleic acid into a virus particle and subsequent transfer into the cell via infection of the cell by the virus particle.

As used herein, the term “polypeptide” encompasses both peptides and proteins, unless indicated otherwise.

The terms “polypeptide,” “protein,” and “peptide” refer to a chain of covalently linked amino acids. In general, the term “peptide” refers to shorter chains of amino acids (e.g., 2-50 amino acids); however, all three terms overlap with respect to the length of the amino acid chain. Polypeptides, proteins and peptides may comprise naturally occurring amino acids, non-naturally occurring amino acids, or a combination of both. The polypeptides, proteins and peptides may be isolated from sources (e.g., cells or tissues) in which they naturally occur, produced recombinantly in cells in vivo or in vitro or in a test tube in vitro, and/or synthesized chemically. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

The term “fragment,” as applied to a polypeptide or protein of this invention, will be understood to mean an amino acid sequence of reduced length relative to a reference (e.g., full length or “wild type”) polypeptide or amino acid sequence and comprising, consisting essentially of, and/or consisting of an amino acid sequence of contiguous amino acids identical to or substantially similar to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

As used herein, “fragment” also refers to a portion of a periostin protein that retains at least one biological activity normally associated with periostin and can have at least about 50% 60%, 65%, 70%, 75%, 80%, 85%, 90% 95% or more of the biological activity as compared with the full-length (e.g., reference) protein or even has a greater level of biological activity.

The term “domain” as used herein is intended to encompass a part of a protein sequence and structure that can evolve, function and exist independently of the rest of the protein chain. A domain is capable of forming a compact three-dimensional structure and often can be independently stable and folded. One domain may appear in a variety of evolutionarily related proteins. Domains can vary in length from between about 25 amino acids up to about 500 amino acids in length. A “domain” can also encompass a domain from a wild-type protein that has had an amino, acid residue, or residues, replaced by conservative substitution. Because they are self-stable in a protein milieu, domains can be “swapped” by genetic engineering between one protein and another to make chimeric proteins.

The terms “variant” or “variants,” as used herein, are intended to designate periostin having the “wild type” or “parent” amino acid sequence (e.g., as provided under the GenBank® Accession numbers provided herein), wherein one or more amino acids of the parent sequence have been substituted by another amino acid and/or wherein one or more amino acids of the parent sequence have been deleted and/or wherein one or more amino acids have been inserted in the parent sequence protein and/or wherein one or more amino acids have been added to the parent sequence. Such addition can take place either at the N-terminal end or at the C-terminal end of the parent protein or both and/or in the interior of the sequence. The “variant” or “variants” within this definition still have periostin activity in their activated form. In one embodiment, a variant is at least 70% identical with the wild type or parent amino acid sequence of periostin. In some embodiments a variant is at least 70%, 75%, 80%, 85, 90%, or 95% identical with the amino acid sequence of periostin. In other embodiments a variant is at least 90% identical with the amino acid sequence of periostin. In a further embodiment a variant is at least 95%, 96%, 97%, 98%, or 99% identical with the amino acid sequence of periostin.

The variant may have “conservative” changes, wherein a substituted amino acid has similar structural or chemical properties. In particular, such changes can be guided by known similarities between amino acids in physical features such as charge density, hydrophobicity/hydrophilicity, size and configuration, so that amino acids are substituted with other amino acids having essentially the same functional properties. For example: Ala may be replaced with Val or Ser; Val may be replaced with Ala, Leu, Met, or Ile, preferably Ala or Leu; Leu may be replaced with Ala, Val or Ile, preferably Val or Ile; Gly may be replaced with Pro or Cys, preferably Pro; Pro may be replaced with Gly, Cys, Ser, or Met, preferably Gly, Cys, or Ser; Cys may be replaced with Gly, Pro, Ser, or Met, preferably Pro or Met; Met may be replaced with Pro or Cys, preferably Cys; His may be replaced with Phe or Gln, preferably Phe; Phe may be replaced with His, Tyr, or Trp, preferably His or Tyr; Tyr may be replaced with His, Phe or Trp, preferably Phe or Trp; Trp may be replaced with Phe or Tyr, preferably Tyr; Asn may be replaced with Gln or Ser, preferably Gln; Gln may be replaced with His, Lys, Glu, Asn, or Ser, preferably Asn or Ser; Ser may be replaced with Gln, Thr, Pro, Cys or Ala; Thr may be replaced with Gln or Ser, preferably Ser; Lys may be replaced with Gln or Arg; Arg may be replaced with Lys, Asp or Glu, preferably Lys or Asp; Asp may be replaced with Lys, Arg, or Glu, preferably Arg or Glu; and Glu may be replaced with Arg or Asp, preferably Asp. Once made, changes can be routinely screened to determine their effects on function.

Alternatively, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, such as for example, LASERGENE™ software. In particular embodiments, a “functional variant” retains at least one biological activity normally associated with periostin. In particular embodiments, the “functional variant” retains at least about 40%, 50%, 60%, 75%, 85%, 90%, 95%, 97%, 98% or more biological activity normally associated with periostin.

As used herein, “derivative” refers to a component that has been subjected to a chemical modification. For example, derivatization of a protein component can involve the replacement of a hydrogen by an acetyl, acyl, alkyl, amino, formyl, or morpholino group. Derivative molecules can retain the biological activities of the naturally occurring molecules but can confer advantages such as longer lifespan and/or enhanced activity.

In particular embodiments, a biologically active variant or derivative of any of the protein components of this invention has at least about 60%, 65%, 70%, 75%, 80%, 85%, 90%, 95%, 96%, 97%, 98%, 99% or more amino acid sequence similarity or identity with the amino acid sequence of a naturally-occurring protein.

A domain or fragment of a polypeptide or protein of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention (e.g., promoting and/or accelerating healing of bone fracture and/or tendon and/or ligament injury or damage) according to the methods described herein, which are routine methods for testing activities of polypeptides, and/or according to any art-known and routine methods for identifying such activities. Such production and testing to identify biologically active fragments of the polypeptides described herein would be well within the scope of one of ordinary skill in the art and would be routine.

The invention further provides homologues, as well as methods of obtaining homologues, of the polypeptides and/or fragments of this invention from other organisms. As used herein, an amino acid sequence or protein is defined as a homologue of a polypeptide or fragment of the present invention if it shares significant homology or identity to a polypeptide, peptide and/or fragment of the present invention. Significant homology or identity means at least 60%. 65%, 70-%, 75%, 80%, 85%, 90%, 95%, 97%, 98%, 99%, and/or 100% homology or identity with another amino acid sequence. In some embodiments, by using the nucleic acids that encode the periostin proteins, peptides and/or fragments of this invention (as are known in the art and incorporated by reference herein), as a probe or primer, and techniques such as PCR amplification and colony/plaque hybridization, one skilled in the art can identify homologues of the periostin polypeptides, peptides and/or fragments of this invention in other organisms on the basis of information available in the art.

A subject of this invention is any subject that is susceptible to bone fracture or injury, as well as injury or damage to a tendon and/or ligament. Nonlimiting examples of a subject of this invention include mammals, such as humans, nonhuman primates, domesticated mammals (e.g., dogs, cats, rabbits), laboratory animals (e.g., mice, rats and other rodents), livestock and agricultural mammals (e.g., horses, cows, pigs).

A subject of this invention can be “in need of” the methods of the present invention, e.g., because the subject has, or is believed at risk for, a disorder including those described herein and/or is a subject that would benefit from the methods of this invention. For example, a subject in need of the methods of this invention can be, but is not limited to, a subject diagnosed with, having or suspected to have, or at risk of having or developing a bone fracture. A subject in need of the methods of this invention can also be, but is not limited to, a subject diagnosed with, having or suspected to have, or at risk of having or developing a ligament and/or tendon injury, damage, trauma and/or irregularity, as well as a subject in need of repair and/or healing of a ligament and/or tendon (e.g., due to surgery, trauma, etc.).

The term “percent identity,” as known in the art, describes a relationship between two or more polypeptide sequences or two or more polynucleotide sequences, as determined by comparing the sequences. In the art, “identity” also means the degree of sequence relatedness between polypeptide or polynucleotide sequences as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

Exemplary methods to determine identity are designed to give the best match between the sequences tested. Methods to determine identity and similarity are codified in publicly available computer programs. Sequence alignments and percent identity calculations can be performed using the Megalign program of the LASERGENE bioinformatics computing suite (DNASTAR Inc., Madison, Wis.). Multiple alignments of sequences may be performed using the Clustal method of alignment (Higgins and Sharp (1989) CABIOS 5:151-153), with the default parameters (GAP PENALTY=10, GAP LENGTH PENALTY=10). Exemplary default parameters for pairwise alignments using the Clustal method can be selected: KTUPLE 1, GAP PENALTY=3, WINDOW=5 and DIAGONALS SAVED=5.

The term “sequence analysis software” refers to any computer algorithm or software program that is useful for the analysis of nucleotide and/or amino acid sequences. “Sequence analysis software” is commercially available or can be independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, the results of the analysis will be based on the “default values” of the program referenced, unless otherwise specified. As used herein “default values” will mean any set of values or parameters, which originally load with the software when first initialized.

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

A “therapeutic polypeptide,” “therapeutic peptide” or “therapeutic fragment” is a polypeptide, peptide or fragment that can alleviate or reduce symptoms that result from an absence or defect or deficiency in a protein in a cell or subject. Alternatively, a “therapeutic polypeptide,” “therapeutic peptide” or “therapeutic fragment” is a polypeptide, peptide or fragment that otherwise confers a benefit to a subject, e.g., by increasing bone development, decreasing bone fracture healing time and/or stimulating and/or enhancing ligament and/or tendon healing.

The term “therapeutically effective amount” or “effective amount,” as used herein, refers to that amount of a polypeptide, peptide, fragment, nucleic acid, virus and/or composition of this invention that imparts a modulating effect, which, for example, can be a beneficial effect, to a subject afflicted with a condition (e.g., a disorder, disease, syndrome, illness, injury, traumatic and/or surgical wound), including improvement in the condition of the subject (e.g., in one or more symptoms), delay or reduction in the progression of the condition, prevention or delay of the onset of the condition, and/or change in clinical parameters, status or classification of a disease or illness, etc., as would be well known in the art.

For example, a therapeutically effective amount or effective amount can refer to the amount of a polypeptide, peptide, fragment, nucleic acid, virus, composition, compound and/or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat” or “treating” or “treatment” refers to any type of action that imparts a modulating effect, which, for example, can be a beneficial effect, to a subject afflicted with a condition (e.g., disorder, disease, syndrome, illness, traumatic or surgical wound, injury, etc.), including improvement in the condition of the subject (e.g., in one or more symptoms), delay or reduction in the progression of the condition, prevention or delay of the onset of the condition, and/or change in clinical parameters, disease or illness, etc., as would be well known in the art.

By the terms “treat,” “treating” or “treatment of” (or grammatically equivalent terms), it is also meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder. In certain embodiments, the methods of this invention can be employed to promote and/or accelerate healing of a bone fracture and/or stimulate and/or enhance ligament and/or tendon healing.

By “prevent,” “preventing” or “prevention” is meant to avoid or eliminate the development and/or manifestation of a pathological state and/or disease condition or status in a subject.

In particular embodiments, the present invention provides a composition comprising, consisting essentially of and/or consisting of a protein, peptide, fragment nucleic acid and/or virus of this invention in a pharmaceutically acceptable carrier and, optionally, further comprising other medicinal agents, pharmaceutical agents, stabilizing agents, buffers, carriers, adjuvants, diluents, etc.

In some embodiments, a composition of this invention can comprise, consist essentially of and/or consist of a protein, peptide, fragment, nucleic acid and/or virus of this invention in combination with an anti-inflammatory agent, a cytokine, an immune modulator and/or a locally acting analgesic (e.g., lidocaine). In some embodiments, a composition of this invention can comprise, consist essentially of and/or consist of a protein, peptide, fragment, nucleic acid and/or virus of this invention in combination with a nucleic acid encoding an anti-inflammatory agent and/or cytokine of this invention.

Further provided herein is a pharmaceutical composition comprising a protein, peptide, fragment, nucleic acid and/or virus of this invention in a pharmaceutically acceptable carrier, in any combination.

“Pharmaceutically acceptable,” as used herein, means a material that is not biologically or otherwise undesirable, i.e., the material may be administered to a subject along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; latest edition). Exemplary pharmaceutically acceptable carriers for the compositions of this invention include, but are not limited to, sterile pyrogen-free water and sterile pyrogen-free physiological saline solution, as well as other carriers suitable for injection into and/or delivery to a subject of this invention, particularly a human subject, as would be well known in the art.

A further aspect of the invention is a method of administering or delivering a periostin protein, peptide, fragment, nucleic acid and/or virus of the invention to a subject of this invention. Administration or delivery to a human subject or an animal in need thereof can be by any means known in the art for administering proteins, peptides, fragments, nucleic acids and/or viruses. In some embodiments, the protein, peptide, fragment, nucleic acid and/or virus is delivered in a therapeutically effective dose in a pharmaceutically acceptable carrier.

In embodiments in which a nucleic acid of this invention is delivered in a viral vector (e.g., a virus particle), the dosage of virus particles to be administered to a subject will depend upon the mode of administration, the disease or condition to be treated, the individual subject's condition, the particular virus vector, and the nucleic acid to be delivered, and can be determined in a routine manner. Exemplary doses are virus titers of at least about 10⁵, 10⁶, 10⁷, 10⁸, 10⁹, 10¹⁰, 10¹¹, 10¹², 10³, 10¹⁴, 10¹⁵ transducing units or more, including in some embodiments about 10⁸-10¹³ transducing units and including in yet other embodiments about 10¹² transducing units.

In some embodiments, more than one administration (e.g., two, three, four or more administrations) of the protein, peptide, fragment, nucleic acid and/or viral vector may be employed to achieve the desired level of gene expression over a period of various intervals, e.g., daily, weekly, monthly, yearly, etc.

Exemplary modes of administration of the proteins, peptides, fragments, nucleic acids and/or vectors of this invention can include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular protein, peptide, fragment, nucleic acid and/or vector that is being used.

For injection, the carrier will typically be a liquid. For other methods of administration, the carrier may be either solid or liquid. For inhalation administration, the carrier will be respirable, and will preferably be in solid or liquid particulate form.

As described in the embodiments herein, a protein, peptide, fragment, nucleic acid and/or vector a can be administered directly to an injury and/or trauma and/or surgical site of a subject according to the methods of this invention as described herein. In certain embodiments, the protein, peptide, fragment, nucleic acid and/or virus vector will be present in a pharmaceutical composition further comprising a pharmaceutically acceptable carrier. Nonlimiting examples of various modes of administration of the protein, peptide, fragment, nucleic acid and/or virus vector of this invention include the following, singly and/or in any combination.
1. Periostin and/or peptides and/or fragments diluted in vehicle directly administered to injury/trauma/surgical/wound site.
2. Periostin and/or peptides and/or fragments, combined with natural and synthetic hydrogels, directly administered to injury/trauma/surgical/wound site.
3. Periostin and/or peptides and/or fragments in a composition as a paste, putty and/or slurry with demineralized bone matrix, directly administered to injury/trauma/surgical/wound site.
4. Viral delivery of nucleic acid encoding periostin and/or peptides and/or fragments administered directly to the injury/trauma/surgical/wound site in a hydrogel and/or organic sponge and/or implanatable matrix or scaffold and/or individual bolus.
5. Periostin, peptides and/or fragments incorporated into an organic sponge and/or implantable matrix or scaffold and delivered to the injury/trauma/surgical/wound site.
6. Direct injection of virus particles comprising nucleic acid encoding periostin and/or periostin peptides and/or fragments into bone marrow stem cells that serve as a delivery vehicle to the injury/trauma/surgical/wound/fracture/damage site.
7. Addition of periostin, peptides and/or fragments to bone chips, either allograft or autograft.
8. Intravenous delivery of periostin, peptides and/or fragments.
9. Oral delivery of periostin, peptides and/or fragments.
10. Transdermal delivery of periostin and/or peptides and/or fragments.

In some embodiments, the implantable matrix or scaffold can comprise, consist essentially of, and/or consist of an implantable device, a surgical graft material, a positively-charged nylon membrane, a suture, cat gut, a tissue scaffold, or a bone graft substitute or any combination thereof. In certain embodiments, the implantable matrix can comprise, consist essentially of and/or consist of polytetrafluoroethylene (GORTEX™), poliglecaprone (MONOCRYL™), high density polyethylene (MARLEX™), polypropylene, polyglactin, polydiaxanone (PDS), or polyethylene terephthalate (DACRON™), as described in U.S. Pat. No. 7,201,898, the entire contents of which are incorporated by reference herein.

Dosages of the periostin protein, peptides and/or active fragment thereof to be administered to a subject will depend upon the mode of administration, the disease or condition to be treated, the individual subject's condition, the particular protein, peptide and/or fragment or nucleic acid encoding same, and any other agents being administered to the subject and can be determined in a routine manner according to methods well known in the art. An exemplary dosage range for a human subject is from about 1 microgram/ml of vehicle to about 500 milligrams/ml of vehicle

In particular embodiments, more than one administration (e.g., two, three, four or more administrations) of the protein, peptide, fragment and/or nucleic acid of this invention may be employed to achieve the desired result over a period of various intervals, e.g., daily, weekly, monthly, yearly, etc.

The present invention will now be described with reference to the following examples. It should be appreciated that these examples are for the purposes of illustrating aspects of the present invention, and do not limit the scope of the invention as defined by the claims.

### Examples

### Example I

### Periostin Studies

More than 28 million musculoskeletal injuries occur per year in the US. Some of these bone fractures heal well, yet can take as long as 12 weeks. Many require surgery in order to facilitate the healing process using one of three methods: mechanical stabilization, addition of biological therapeutics (e.g., BMP), or a combination of mechanical and biological aids. In spite of this, 5-10% of all bone fractures still do not heal by three months, termed fracture non-unions. Often severe trauma with bone loss is the reason for non-unions, but the overall health of the patient also dramatically impacts bone fracture healing. For example, diabetic or osteoporetic patients with bone fractures are at a much higher risk of non-union. Thus, finding new biological therapeutics that promote fracture healing, would greatly reduce the morbidity and mortality that result from bone injuries.

### Fracture Repair

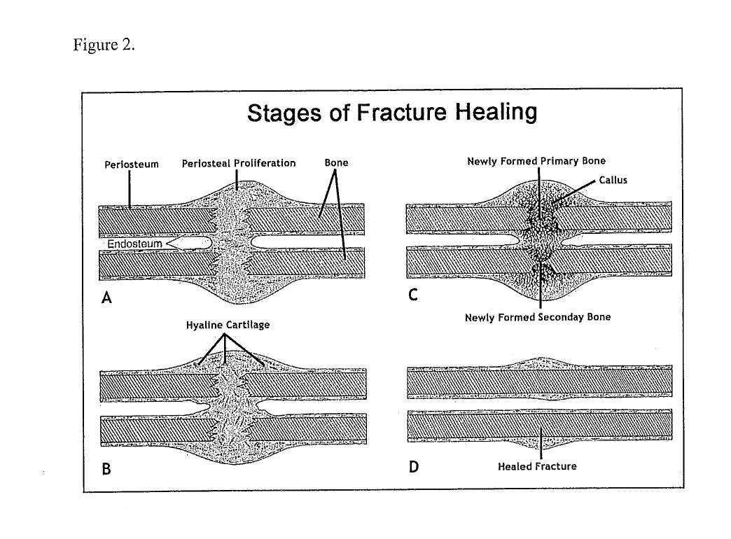

Following fracture, bone matrix is destroyed and cells adjoining the fracture site die. Damaged blood vessels produce a localized hemorrhage resulting in the formation of blood clots. These clots, as well as cytokines released from the dead cells, induce an initial inflammatory/immune response, stimulating macrophage mobilization to the wound site. Within hours to days following fracture, macrophage excavation of the dead tissue is completed and the periosteal and endosteal cells respond by expansive proliferation and migration. This response is necessary to ensheath the fractured area with cells which, in-turn, will promote callus formation. Formation of primary bone is then initiated by local periosteal cells, pre-osteoblasts and bone marrow stem cells, which are stimulated to differentiate into bone forming osteoblasts. These osteoblasts undergo endochondral and intramembranous bone ossification, contributing simultaneously to the healing of the fractured area and maturation of the bony callus. The primary bone of the newly formed callus is eventually remodeled and replaced by secondary tissue which, over-time will regenerate into fully mature bone (FIGS. 2A-D).

### Bone Morphogenetic Proteins (BMPs)

BMPs, originally isolated as proteins capable of inducing bone and cartilage formation, are members of the transforming growth factor-β (TGFβ) superfamily of polypeptides [18]. BMPs are translated as large preproteins composed of a signal sequence, a prodomain, and a mature domain. During secretion, the signal sequence is cleaved and the BMP proprotein undergoes dimerization. Specific enzymes present in the extracellular milieu cleave the proprotein, thereby generating a mature, active BMP dimer protein. The dimer can bind to a host of cell surface receptors and initiate a myriad of intracellular cascades. Through these transmitted signals, BMPs have been shown to induce chemotaxis, migration, proliferation, and differentiation of mesenchymal stem cells [19]. Due to the ability for specific BMP molecules (BMP-2, and BMP-7) to function as osteoinductive molecules capable of inducing bone formation in vitro, extensive research has focused primarily on these proteins as therapeutic regimes for treating bone fractures. As such, several groups have demonstrated the ability of recombinant human BMP-2 (rhBMP-2) and rhBMP-7 to heal critically-sized defects (defined as the smallest intraosseous wound that would not heal by endogenous bone formation alone) in the rat femur, rabbit ulna and canine ulna [19]. In human studies, application of these proteins has been shown in many cases to promote bone healing [20]. The mechanisms by which these BMPs can stimulate bone healing have been speculative at best. However, it is understood that BMPs, acting as signaling molecules, function primarily as stimulants, regulating a variety of cellular processes including migration, proliferation and differentiation, each of which is critical for the promotion of bone healing. Due to the multi-functionality of BMP molecules and their pleiotrophic affects on a myriad of pathways, teasing apart their mechanism(s) of action is prohibitively difficult. Additionally, because BMPs can bind and interact with a variety of receptors, thus altering a vast array of downstream targets, their affect during bone healing may be compromised or diluted.

### Periostin

Previous studies have shown that periostin (i) is stimulated by BMP-2 [21], (ii) expression is significantly stimulated following injury [22], (iii) promotes differentiation of progenitor cells into fibroblasts [23], (iv) regulates collagen fibrillogenesis [24], (v) is required for the biomechanical properties of connective tissues [24], and (vi) is expressed in its namesake the periosteum, which is know to play a role in bone fracture repair [25,26].

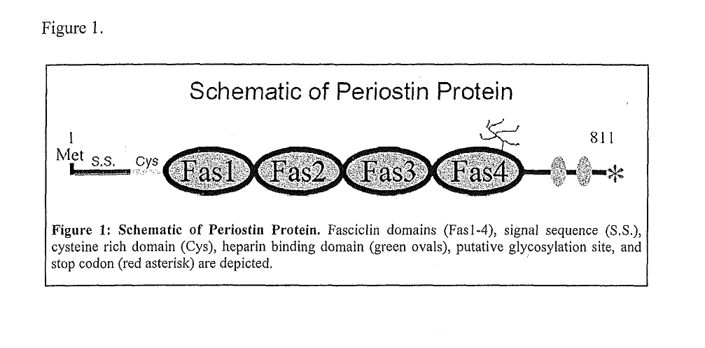

The periostin gene was initially cloned from a mouse calvarial cell line (MC3T3-L1) and shown to promote adhesion and migration of these cells in culture [27]. The encoded protein has a molecular weight of 90 kDa and based on amino acid sequence similarities, is most highly related to the ancestral fasciclin gene in Drosophila. The protein has a signal sequence (targeting it for secretion), four coiled fasciclin like repeats, an amino terminal cysteine rich region and putative heparin binding domains present in the carboxyl tail (FIG. 1). RT-PCR, Western analysis, and genomic sequencing have revealed that at least six carboxyl splice variants may be produced from the periostin locus. It is an evolutionarily conserved protein with chick and zebrafish periostin being 65% homologous to mouse and 70% to human (73% to rat) at the amino acid level [28]. Periostin is one of four known mammalian genes that encode fasiclin domains. The other fasciclin genes are: TGFβ-induced gene-Human clone 3 (a.k.a. βigH3), as well as stabilin 1 and 2. βIG-H3 shares 49% overall amino acid homology (70% homology in the fasiclin domain) with periostin whereas the stabilin proteins are significantly more divergent.

Bornstein and colleagues [18] have proposed that secreted extracellular matrix proteins that function more in regulation of cell matrix interactions than function as structural proteins constitute a related family of proteins, called matricellular proteins. Unlike structural proteins such as collagen, laminins and elastin, matricellular proteins derive their complex functions from their ability to interact with cell-surface receptors (especially integrins), cytokines, growth factors, and/or proteases in addition to structural proteins. Of interest, the expression of this unique family of proteins is most prominent during development and growth, or in response to injury. Examples of matricellular proteins that are defined by their ability to interact with the extracellular matrix and the cell membrane to function both as a structural and signaling molecule during development and disease (injury) include thrombospondins, tenascin-C, osteopontin, CCN1 and SPARC [29].

Periostin has been shown to be capable of interacting with various pairs of integrins in a variety of cell types [30]. Specifically, periostin binding to integrins αᵥ/β₃ and β₁ in mesenchymal cells can initiate signaling related to differentiation, migration and collagen compaction transduced through Rho and PI3 kinases. Even though the exact peptide sequences are not well defined, this integrin binding likely occurs through highly conserved H1 and H2 peptide stretches (but not RGD sequences) present within the fasciclin domain having “YH” and Asp-Ile motifs [30]. In addition to its ability to specifically bind and signal through various integrin receptors, periostin is also able to interact with components of the extracellular space, including collagen I, fibronectin, and tenascin-C. These matrix interactions have been shown to be crucial for (i) promoting collagen fibrillogenesis and (ii) maintaining the biomechanical properties of connective tissues [24]. Thus, based on its known biological roles to date, periostin also appears to qualify as a matricellular protein.

### Periostin Knockout Mice

To examine the function of periostin in vivo, periostin knockout mice were generated [31]. Although the mice are viable and fertile, studies with these animals demonstrate that periostin is essential for proper fibroblast differentiation and collagen production; This is most readily apparent during injury responses and scar formation processes. For example, following an induced myocardial infarction, periostin −/− mice exhibit significantly less fibrosis and collagen deposition that results in enhanced cardiac function [31-33]. In addition, during cardiac valve development, it has been noted that periostin functions as a hierarchical switch, promoting fibroblast differentiation, collagen deposition, collagen cross-linking and is required for maintaining the biomechanical properties of connective tissues [23,34].

Periostin Cooperates with Collagen I to Promote Bone Growth.

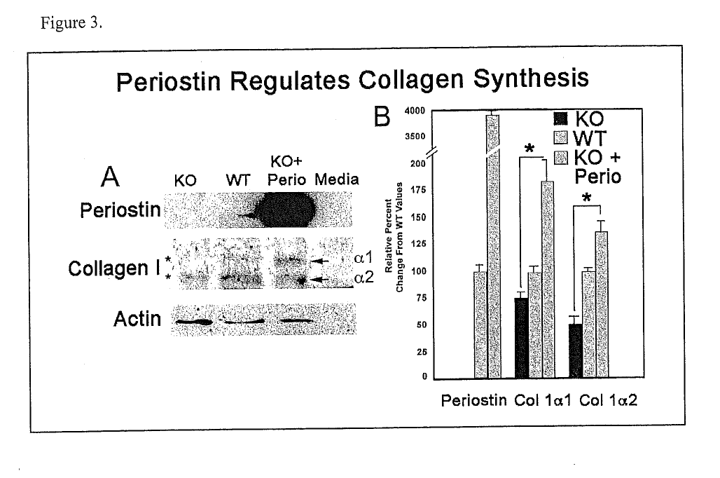

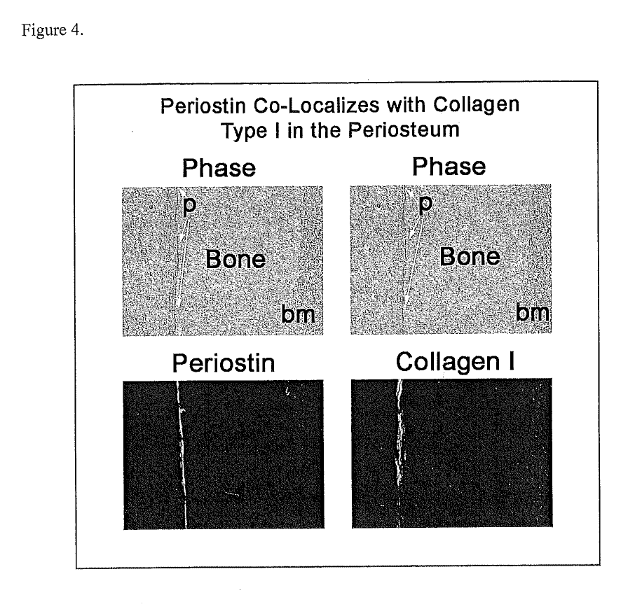

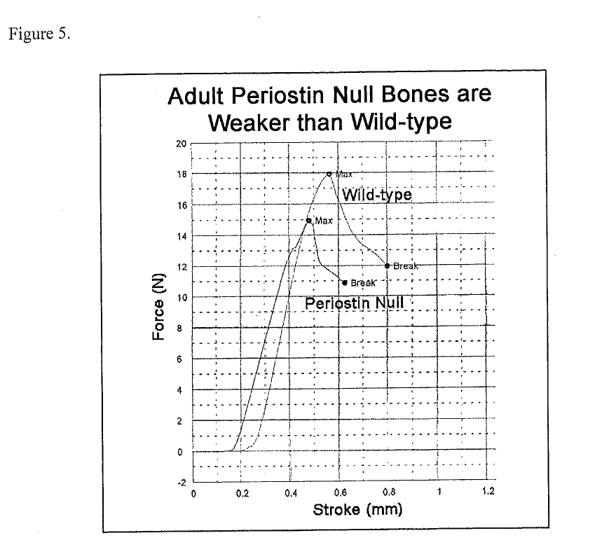

Studies were carried out to examine the affect of periostin on fibroblast differentiation. Through these studies, it was ascertained that periostin was a crucial regulator of collagen I synthesis and maturation (fibrillogenesis) and promoted fibroblast differentiation (FIGS. 3A-B) [23,34]. Further studies focusing on collagen rich tissues, including adult murine bone, demonstrated extensive overlap of expression between collagen I and periostin (FIG. 4). Within the bone, expression of these matrix components was seen primarily in the periosteal cells. To ascertain the functional significance of periostin on bone growth, gene targeted mice were generated. These mice are viable and able to reproduce. However, significant defects have been observed in the material properties of various connective tissues. These defects have been shown to result from alterations in collagen fibrillogenesis [24,35]. For example, (i) TEM (transmission electron microscopy) and morphometric analyses demonstrated reduced collagen fibril diameters in skin dermis of periostin null mice, and (ii) differential scanning calorimetry (DSC) demonstrated a lower collagen denaturing temperature in periostin null mice, reflecting a reduced level of collagen cross-linking [24]. To test the material properties of the adult bone, a three-point bending material testing system (MTS) was used. Data from these experiments indicate that the femurs of the periostin null mice exhibited significantly weaker strength than wild-type mice (FIG. 5).

### Periostin Promotes Bone Fracture Healing.

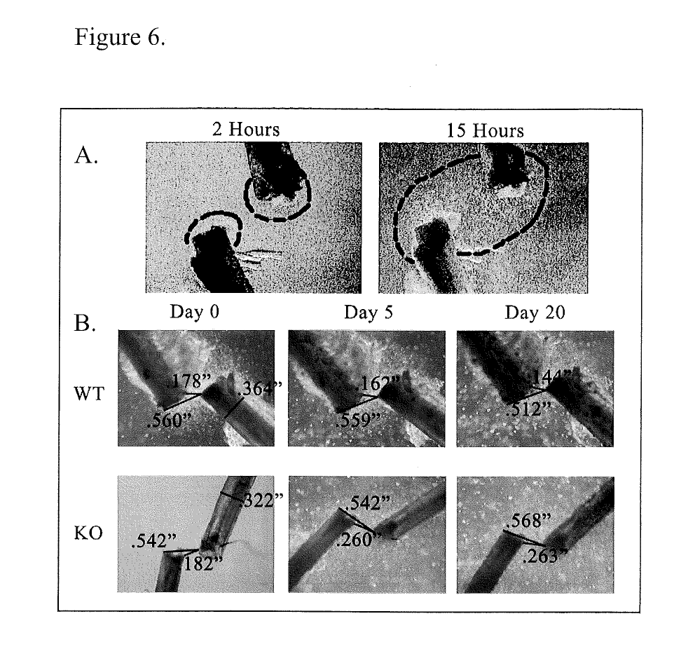

To examine the role of periostin as a mediator of bone healing following injury, a novel in vivo murine fibula osteotomy model was developed (FIGS. 6A-B). The generation of this model system in the mouse has significant advantages over the more standard tibia and femur bone break models. Currently, the main disadvantage of the tibia and femur models is their necessity for an intermedullary stabilizer (i.e., metal rods) due to these bones being weight bearing. These rods make various analyses such microCT, MTS and histological examination either exceedingly difficult or totally impossible. Because the fibula is a non-weight bearing bone, intermedullary stabilization is not required. As such, analyses such as microCT, MTS and histology are more easily and reproducibly performed in the fibula osteotomy model.

To determine the potential role of periostin during the fracture healing process, this osteotomy model was used to generate fibula fractures in wild-type mice.

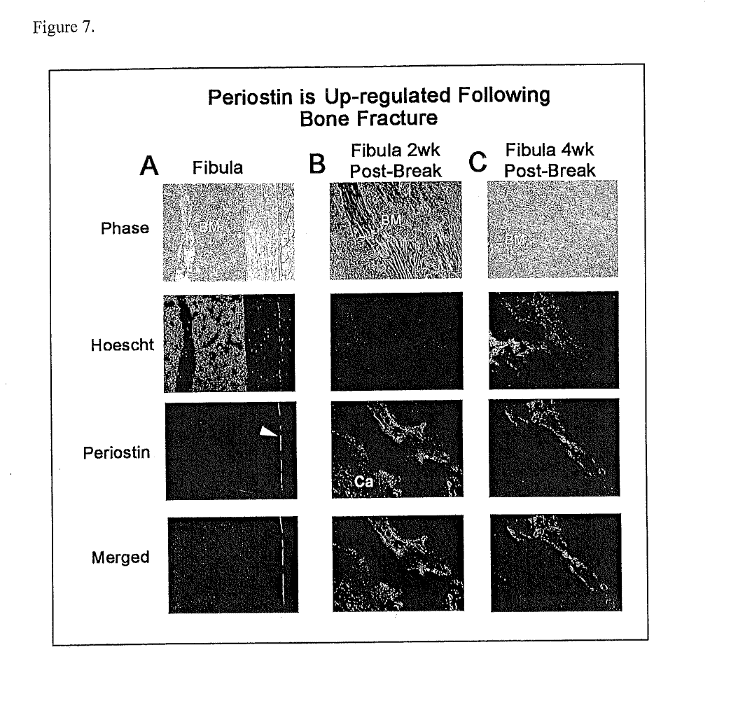

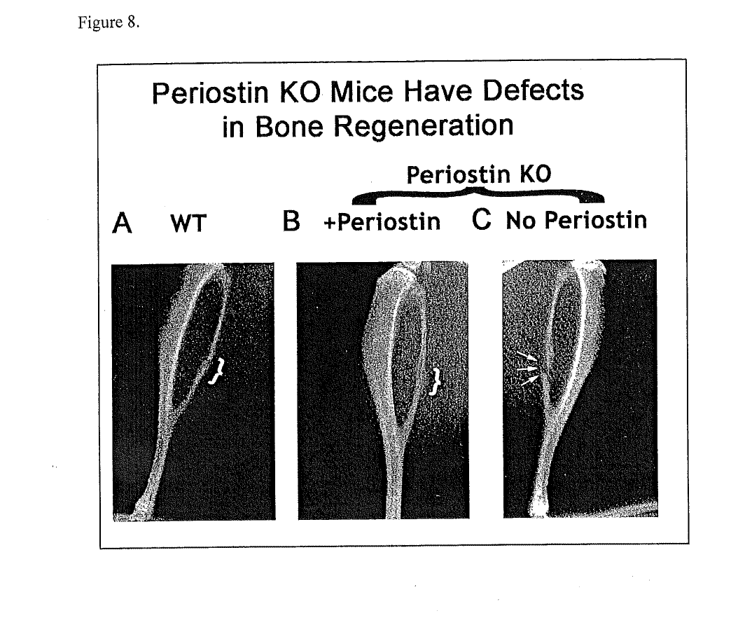

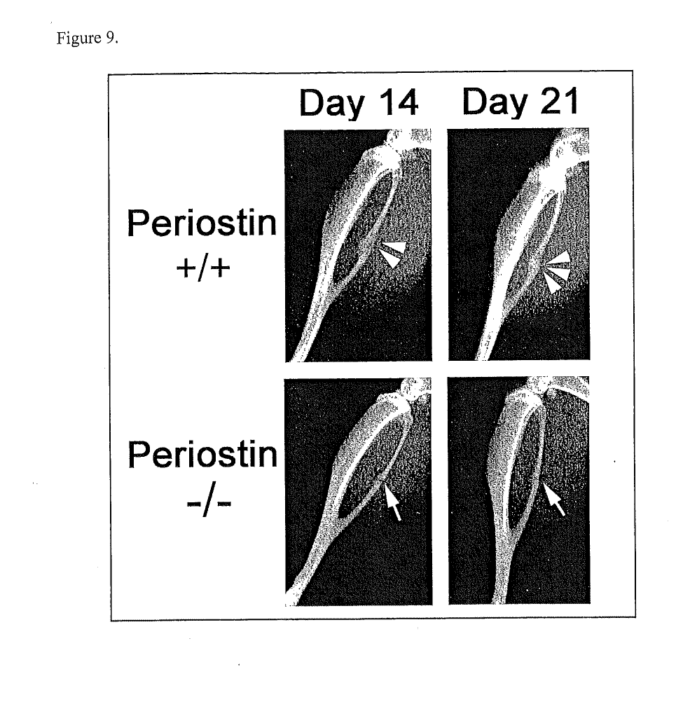

Immunohistochemical analyses indicate that whereas periostin is specifically expressed in the periosteum prior to fracture, this domain of expression greatly expands at the 2 week time-point to include not only the fracture site and forming callus but also the bone marrow cells (FIGS. 7A-C). By the four-week time-point periostin expression remains intense, although more confined to the callus and new bone forming regions while expression has returned to nearly baseline levels in the bone marrow. These data indicate that periostin is a relatively early responder to the injury. This expression profile in and around the fracture indicates that periostin may be functioning to promote bone regeneration. To further examine this, fibula osteotomies were performed in the context of the periostin null mouse. FIG. 9 shows that genetic deletion of periostin delays bone fracture healing. FIGS. 8A-C demonstrate that in the absence of periostin, the fracture bone fails to heal appropriately (FIG. 8C) as compared to the wild-type mouse (FIG. 8A). However, on the opposite leg of the same periostin null mouse, purified periostin protein was exogenously added to the fracture area through a hydrogel delivery format. X-ray analyses demonstrated that the addition of purified periostin “rescued” the null phenotype, showing nice callus formation and refusion of the fractured fibula. These data indicate that periostin is an important, even essential, early mediator of the bone healing process.

### Periostin Promotes Fracture Healing Through Modifying Osteoblast Behavior Following Wounding.

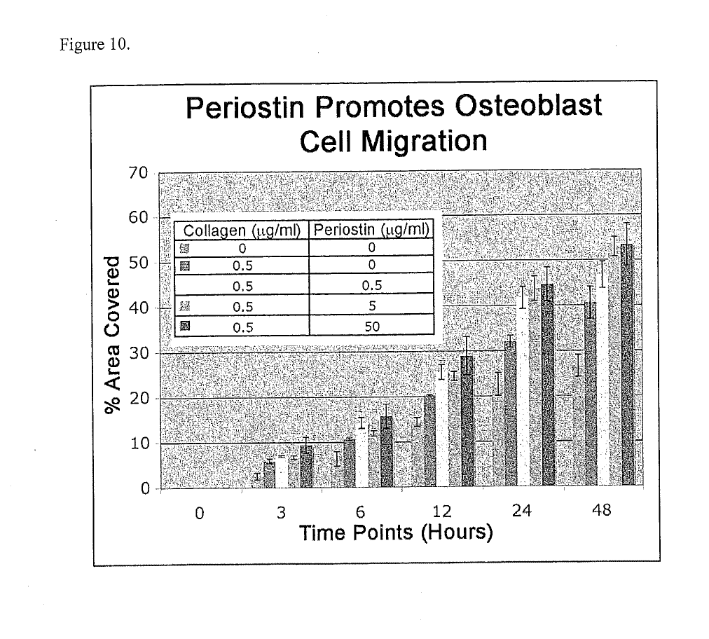

The ability of periostin to promote migration of osteoblast cells in culture was ascertained utilizing a standard “wounding” assay [36]. For this, a monolayer of rat osteoblast cells was plated on either plastic, collagen or collagen plus titrating amounts of periostin. A small scratch was made (with a P200 tip) through the middle of the monolayer and the cells were allowed to migrate into the “wounded” area for up to 48 hours. As FIG. 10 demonstrates, collagen I promotes migration compared to plastic. However, the addition of titrating amounts of periostin plus collagen gave the highest degree of migration, indicating that a collagen/periostin rich matrix would be the best means for promoting osteoblast migration both in vitro and in vivo. These results were compared to those obtained when performing scratch assays in the presence of titrating amounts of BMP-2, which was chosen due to (i) its ability to stimulate periostin expression in various cell and tissue types, and (ii) its reported ability to promote bone regeneration in vivo following fracture. Studies testing the efficacy of BMP-2 to promote migration demonstrated a decrease in migration compared to periostin. However, there did appear to be a significant increase in BMP-2-induced proliferation.

In summary, the data described above demonstrate that periostin is expressed specifically in the periosteum, regulates collagen synthesis, promotes collagen fibrillogenesis, is upregulated following injury, and appears to be required for fracture healing in mice.

### Example II

### Studies to Establish the in vivo Role of Periostin in Regenerating Bone After Fracture

To assess the role of periostin in fracture repair, the experiments will focus on comparing periostin null mice with wild-type in order to (i) define the impact on structural parameters of the bone as well as examine bone marker expression in adult bone in mice lacking periostin, (ii) establish the in vivo effects of the loss of periostin upon bone fracture repair, and (iii) determine if the exogenous application of periostin protein can enhance bone repair in vivo.

Periostin −/− mice will be compared to WT mice using four approaches: histological methods; MicroCT; X-ray, and mechanical testing. The first series of experiments will be performed on non-fractured periostin and WT mouse femurs, to (i) generate a dataset to compare to the data generated during fracture healing and (ii) understand the role of periostin in bone biology before a fracture. The second series of experiments will utilize the fibula osteotomy model on periostin −/− mice compared to periostin +/+ mice to specifically test how the loss of periostin impacts fracture healing. The fibula osteotomy model was developed to allow for histological, MicroCT and mechanical testing. Because the fibula is not a weight bearing bone and does not require mechanical stabilization (i.e., a metal rod) all three of these procedures can be used; otherwise the rod would preclude the use of each of these techniques in a mouse bone. The third series of experiments will utilize this same bone fracture model, the same mouse genotypes, and the same analyses, but will test the addition of exogenous periostin, and periostin fragments and/or peptides to the bone fracture site immediately following injury.

### Comparison of the Expression Analysis of Periostin Protein and Bone Markers Between Adult WT and Periostin Null Femur and Fibula

The analysis of periostin expression in adult WT bone will be determined by immunohistochemistry (IHC) using previously described immunostaining methods and antibodies [37-40]. The hind limbs of WT adult mice ages 4-6 months will be dissected at the knee and separately fixed in 4% paraformaldehyde (PFA) overnight at room temperature, demineralized using EDTA solution (10-fold volume, 2 changes over a 5 day period) and then embedded in paraffin and sectioned. Staining will utilize the previously described periostin antibody and indirect immunofluorescence staining procedure. Positive signal will be detected using laser scanning confocal microscopy. The periostin protein expression pattern will be compared to immunostaining for the following bone and periosteal markers: collagen I (Abcam), osteocalcin (Santa Cruz); Runx2 (Abcam); and Prx1 [37]. The data gathered using this approach will then be compared to a similar analysis using the periostin −/− mice to further characterize the normal pattern of periostin and examine any alterations in the periostin null mouse of known bone marker expression.

MicroCT Analyses of Femur and Fibula from Adult Periostin +/+, +/−, and −/− Mice

At least eight male mice between 4-6 months of age, from each genotype group (periostin +/+, +/− and −/−), will have their hind limbs removed after euthanizing, then both the femur and fibula will be scanned by MicroCT. The images generated by this approach will be analyzed for multiple parameters as described herein [41,42].

Mechanical Testing of Femur from Adult Periostin +/+. +/− and −/− Mice

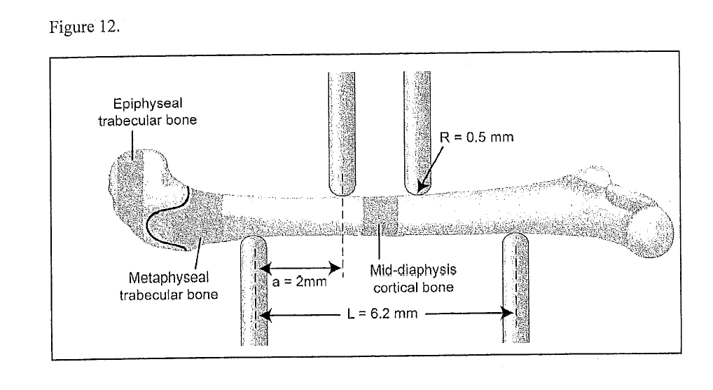

The same bones analyzed above by MicroCT will be cleaned of all the soft tissue and measured for length and weight. Then the bone strength and rigidity will be analyzed using a four point stress test until failure (i.e., breaking) of the bone (FIG. 12). The force applied to the bone will be constantly monitored and the force required to break the bone will be determined, along with multiple parameters of strength and structural integrity which can be extrapolated from the combination of MicroCT and mechanical testing measurements.

Testing the Effects of Loss of Periostin on Bone Fracture Healing in vivo

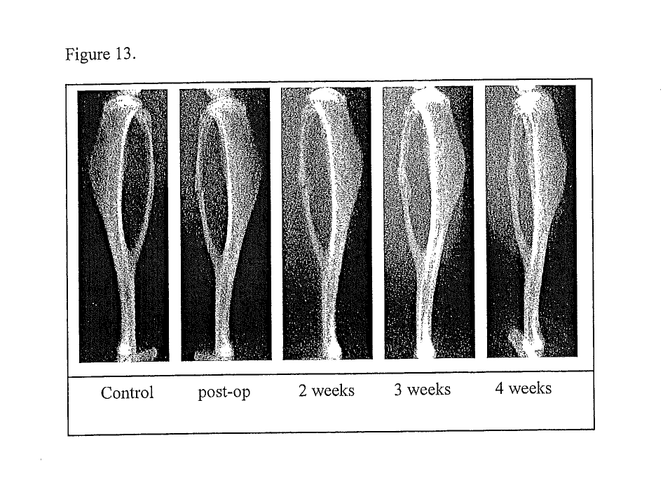

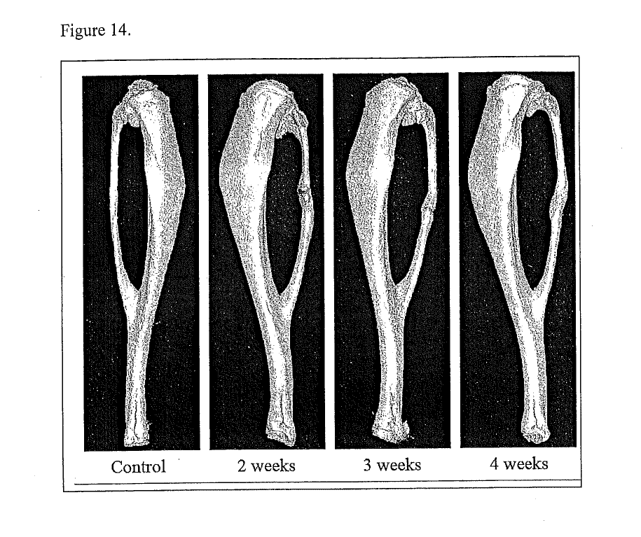

The fibula osteotomy model of bone fracture healing will be used with periostin +/+ and −/− mice ages 4-6 months. Five time points (1, 2, 4, 6, and 8 weeks post fracture) will be examined in the following manner: (i) X-ray (FIG. 13); (ii) MicroCT (FIG. 14); (iii) Histological analysis using Movat's pentachrome and Giemsa stains; and (iv) Immunohistochemistry (IHC) of bone/callus markers (i.e., collagen I (Abcam), collagen II (Abcam), osteocalcin (Sant Cruz), Runx2 (Abcam) and Prx1 [37]. The histological analysis is a terminal analysis for the mouse. However, prior to termination, each mouse will be X-rayed through as many time points as possible until it is euthanized for these terminal histological analyses. This X-ray at every time point for every mouse will generate a documentation of the fracture for each animal and its healing. For each mouse, the right leg will get the fibula osteotomy and the left leg will be sham operated with full incision and suturing, but with no break. There will be a minimum of three mice per time point, multiplied by the two genotypes, which means a total of at least 30 mice will be analyzed in this manner.

### Determination of Whether Exogenous Periostin or Periostin Fragments can Aid in Bone Healing

Full length periostin, periostin peptides and periostin fragments will be tested for the ability to enhance regeneration of bone after fracture. This experiment and all of the analyses will be performed as described above with the exception that for this experiment, both legs will be given a fibula osteotomy. The right leg will be injected with 5 μl of collagen gel (2 μg/ml) with periostin at a concentration of no more than 1 mg/ml of full length periostin in a collagen gel, and the left leg will be injected with the same volume of gel but with no periostin protein. There will be a minimum of three mice per time point.

### Testing of Periostin Peptides or Fragments for Enhancement of Regeneration of Bone After Fracture.

This experiment and all of the analyses will be performed as described above with the exception that for this experiment both legs will be given a fibula osteotomy. The right leg will be injected with 5 μl of collagen gel (2 μg/ml) with a concentration of no more than 1 mg/ml of periostin peptide or fragment in a collagen gel, and the left leg will be injected with the gel but with no periostin peptide or fragment. The efficacy of the peptides and/or fragments will be first evaluated in the periostin −/− mice to see if they can rescue the fracture healing, then they will be evaluated in the +/+ mice to determine if they can further enhance healing (i.e., shorten the healing time). They will be compared to equimolar amounts of periostin full length protein. There will be a minimum of three mice per time point, multiplied by the two genotypes, which means a total of at least 30 mice will be analyzed in this manner.

Some experiments for optimization of gel delivery of periostin into the fracture site have been carried out. Lyophilized periostin protein (R&D Systems calls periostin by its alias, OSF-2) is reconstituted at 5 μl of PBS, which is then added to 4 μl of collagen gel, mixed and all 5 μl added to the fracture site. Studies to determine the best delivery options will be carried out, which may include slower or faster release gels. Other options include hyaluronan based gels (Glycosan) and Pluronic gel.

An alternative to the fibula osteotomy model is a drilled femur model. A hole is drilled in the femur, in the metaphyseal region close to the hip joint, with a 0.55 mm drill bit. It is not pushed through the entire bone but only creates an injury/hole on one side. It produces a very specific bone injury with a very specific size and is therefore very reproducible.

### Example III

### Studies to Determine, in vitro, the Role of Periostin in Promoting Cellular Changes/Responses Necessary for Bone Regeneration

The in vitro assays described herein will be used to initially screen a large number of periostin peptides and fragments. Once the peptide(s) and/or fragment(s) is/are identified that can impart appropriate cell adhesion and migration (i.e., similar to full length periostin) then they will be further tested for their ability to promote fracture healing in vivo.

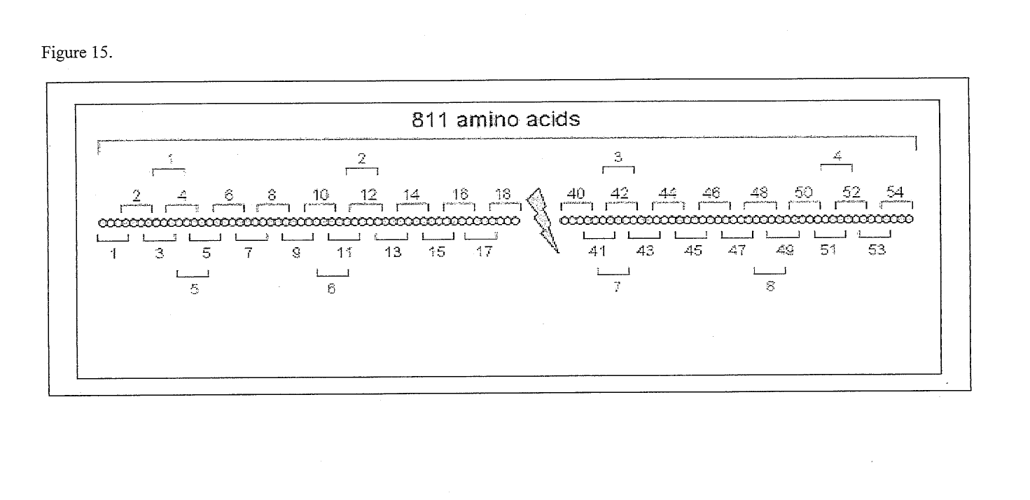

All assays will utilize the following primary cells and cell lines that have previously been demonstrated as the most applicable model systems for studying osteogenesis in vitro. ROS 17/2.8 is a rat cell line derived from an osteosarcoma that will be used as a model of osteoblasts [43]; MC3T3-E1 (subclone 4) is a mouse derived pre-osteoblast cell line that will be used as a model of cells that can develop into osteoblasts [44]; and primary mouse calvarial osteoblasts will be used as a model of non transformed osteoblasts [45]. These cells will be evaluated in assays that assess cell adhesion, migration, and invasion, which are all important cell behaviors typical of bone regeneration after fracture. An important feature of the in vitro assays is to define the region (e.g., peptide or fragment or domain) of the periostin protein that contains the bioactivity essential for bone regeneration. In particular embodiments, this will be performed by using more than 55 synthesized overlapping peptides, each ˜20 amino acids in length from the periostin protein (FIG. 15).

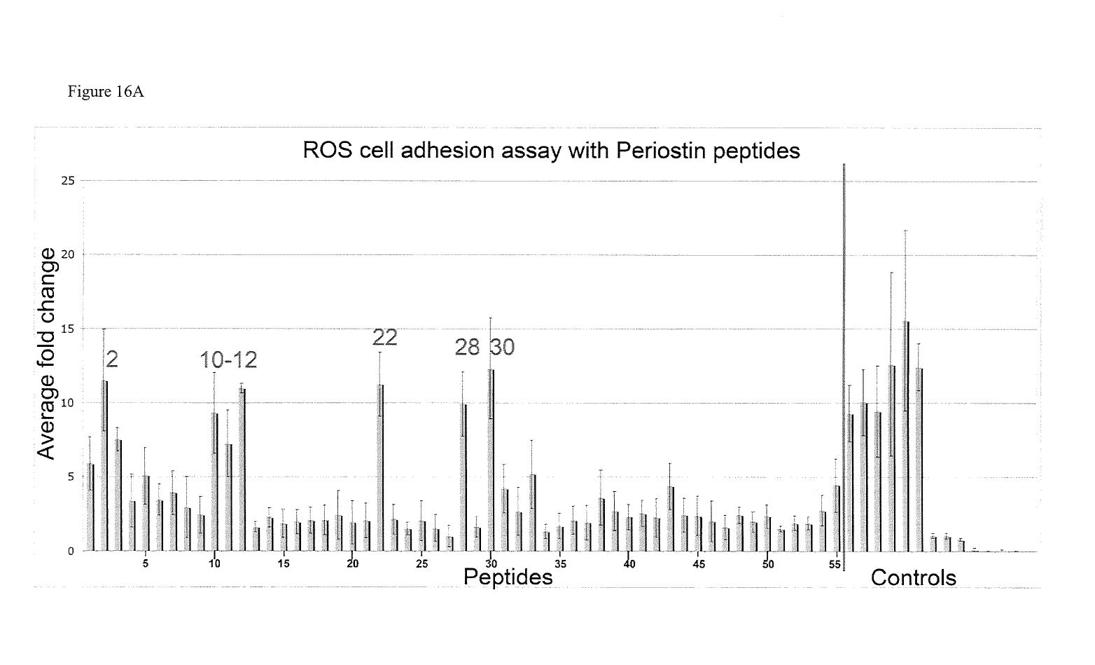

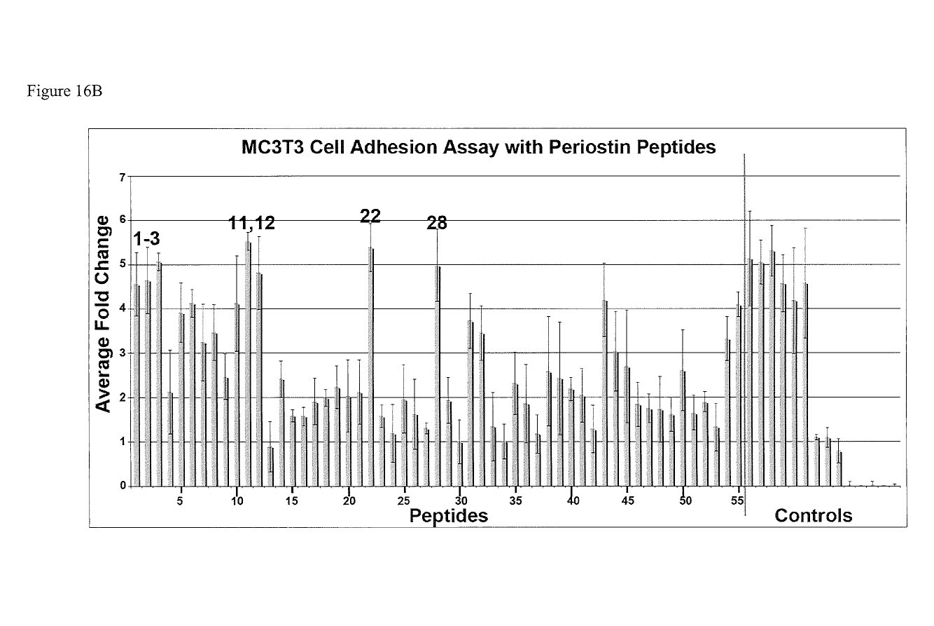

The 55 peptides in Table 1 have been studied in a cell adhesion assay using the ROS cell line. Peptides 2, 10, 11, 12, 22, 28 and 30 showed significant binding (FIG. 16).

Studies to Test the Ability of Various Bone Related Cells to Migrate on, or Invade into, Various Matrices Containing Periostin or Periostin Fragments

The analysis of the three models of bone related cells will be evaluated in both 2-D and 3-D migrations assays. The 2-D or “scratch” assay will be performed by plating the cells at near-confluency 24 hours prior to the “scratch” in medium with 1.5% serum. The “scratch” is produced with a 200 μl pipette tip, the cells are washed gently with PBS, and then medium is added to the cells but the medium does not contain serum. The serum is minimized in the setup and after the scratch to minimize its proliferative effects on cells that could confound the determination of migration. The proliferation is assessed by using a 4 hour pulse of 10 μM BrdU and immunolabeling [46] with a FITC-conjugated anti-BrdU antibody (Abcam) or immunostaining for PCNA [47]. The minimum amount of serum may need to be determined for each cell type so that the cells remain viable but not proliferative. The “scratch” is marked at three discrete locations so that it can be digitally captured at exactly the same position over the time points of 0, 3, 6, 12, 24, and 48 hours after scratching the confluent monolayer. The surface area of the scratch on the digital images is measured using a Photoshop Creative Suite 4 program. Three different fields of the same scratch are analyzed per well of a 12 well dish and three wells are used per condition, with the results being averaged together and statistically analyzed. The migration of cells on various matrices (e.g., collagen, periostin, collagen+periostin, peptides, etc.) can be assessed relatively quickly with this assay.

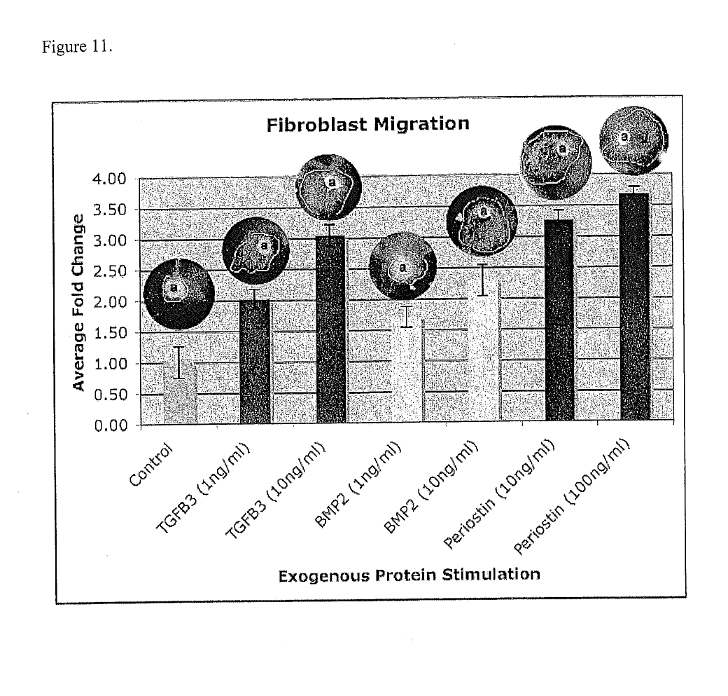

The 3-D migration and invasion assay will use the same cells but aggregate them using a hanging drop method overnight. The following day, the cells are placed on top of a collagen gel (2 mg/ml) and incubated for 72 hours, after which the gels and cells are fixed in 4% paraformaldehyde and can be immunostained or chemically stained prior to digital capture and morphometric analysis. The number of cells that have migrated out from the aggregate and the distance migrated on the top of the gel are the first order parameters of migration. FIG. 11 shows the results of migration studies done with the hanging drop method.

A second order is to analyze the cells that invade into the gel and measure how deeply they penetrate. At least three aggregates will be scored in these ways, averaged together and then statistically analyzed. The collagen gels can be supplemented with other proteins, specifically full-length periostin or periostin peptides or fragments. The peptides and/or fragments will be compared singly and in combinations to the full length periostin to determine if one or more of them contain the same level of bioactivity as the full length protein.

Studies to Test the Ability of Various Bone Related Cells to Adhere to Matrices Containing Periostin or Periostin Peptides and/or Fragments

The analysis of the three models of bone related cells will be evaluated in terms of their adhesion as described [30]. Adhesion of the cells to various matrices such as collagen, periostin, periostin peptides, periostin fragments and/or combinations thereof, will be investigated. After the adhesion assays are completed, the numbers for each triplicate are averaged, and the dilutions are plotted on a graph to assess if the binding is dependent on amount of substrate and how it compares from one substrate to another. As a control experiment, peptides and/or fragments that are identified will be verified for cell binding by incubating the cells with the candidate peptides and/or fragments prior to plating on a full-length periostin substrate.

Studies to Test which Integrin Receptor Binds to Periostin and where this Site is Located on the Periostin Protein

The adhesion assay described herein will be used in the same way here with the exception that periostin will be the only matrix analyzed, and its binding will be blocked by antibodies to a panel of specific integrins as described [30]. To assess the signaling pathway that is likely impacted by periostin/integrin binding, pharmacologic inhibition using small molecule inhibitors Y-27632 (Calbiochem, 5 μM) or wortmannin (Calbiochem, 1 μM), to block Rho-kinase and PI-3 kinase, respectively, will be tested. Once it is determined which integrin receptor is being used on the various cells to bind periostin, the specific region of periostin (utilizing the peptides and/or fragments) will be defined. Antibody blocking will be used to confirm that this peptide-cell interaction is via a specific integrin.

### Cell Adhesion Assay

These assays will be performed as described [30]. Briefly, titrated amounts of purified matrix protein (or peptides or fragments) ranging from 10 ng/ml to 100 μg/ml will be used to coat the wells of a 96-well dish. Poly-L-lysine (1.5 μg/ml) and 1% BSA will be used as positive and negative controls for adhesion, respectively. The wells are blocked with 1% BSA to coat any of the charged plastic surface not coated by the substrates. Finally the cells are allowed to adhere to the substrates at 37° C. for 1-2 hours depending on cell type. The cells are then gently washed to remove those that are not adherent and retain those that are adherent. Then the cells remaining are exposed to 4% PFA to fix them and they are subsequently stained with 0.25% crystal violet for 30 min. Plates are washed and air-dried. At this point the cells can be visualized and images digitally captured. The amount of stain on the cells is solubilized by 2% sodium deoxycholate for 10 min. and absorbance is measured at 540 nm wavelength. This gives quantitative data directly related to the number of adherent cells. Another assessment can be done by digitally capturing the cells in each well and analyzing their spreading by morphometric analysis.

### Migration Assays

These assays will be as described herein for 2-D migration assays [30,36]

### Histology/IHC

All histological techniques, stains and antibodies are routine and most are standard and even commercially available. Movats pentachrome stain and Giemsa stain are both commercially available. The antibodies that will be used are all purchased except for Prx1 [37]. These antibodies are all directed against proteins that are specific for bone, fractured callus, or periosteal regions. The Prx1 protein is expressed in the periosteal region in the developing embryo [37] and in the adult.

### Periostin and Periostin Peptides

Full length recombinant mouse periostin (a.k.a. OSF-2) is purchased from R&D systems. The peptides in Table 1 have been purchased and are 20 amino acids in length with 5 amino acid overlaps.

### Cell Lines and Primary Cell Cultures

Three different models of bone related cells in culture will be used: (i) ROS 17/2.8 is a rat cell line derived from an osteosarcoma that will be used as a model of osteoblasts [43]; (ii) MC3T3-E1 subclone 4 is a mouse derived pre-osteoblast cell line that will be used as a model of cells that can develop into osteoblasts [44]; and (iii) primary mouse calvarial osteoblasts will be used as a model of non transformed osteoblasts [45].

### Bone Fracture (Fibula Osteotomy Model)

After full body anesthesia using an intraperitoneal injection of 0.4% chloral hydrate (1 cc/100 g body weight) and full sterilization of the leg, the mid-shaft of the fibula is exposed via a posterior-lateral approach. Following fibula osteotomy with small surgical scissors, the tissue is all closed using two interrupted sutures of Ethicon Vicryl 5.0. This permits full weight bearing by the mice after anesthesia wears off.

### Micro-CT Imaging

The distal portion and middle portion of the femur will be scanned by micro-CT (Inveon CT, Siemens Medical Solutions, Knoxville, Tenn.) and analyzed as previously performed [41,42]. The morphological indices of bone volume and architecture will be determined in the epiphyseal, metaphyseal and diaphyseal regions of the femur. The trabecular volume of interest in the epiphysis will contain a 0.32 mm section with the most distal slice defined as the plane where the trabecular bone of the condyles connected. The metaphyseal region will include a 0.80 mm section with the first slice starting right after the last sign of growth plate in the center of the femur. The trabecular bone will be isolated from the cortical bone by visually drawing the volume of interest (VOI). Cortical bone will be analyzed from a 1 mm section located in the mid-diaphysis, which will be the same region of the femur that would later be broken in mechanical testing.

Bone tissue will be segmented from the marrow and soft tissue using a thresholding procedure. The epiphyseal and metaphyseal trabecular bone will be analyzed separately for bone volume fraction (BVF), bone mineral density (BMD), and trabecular number, thickness and spacing, as well as the total volume of each VOI. For the mid-shaft, the bone volume in the 1 mm section, as well as the cortical thickness and BMD, will be calculated directly with the micro-CT software. In order to determine the geometry of the cortical bone in diaphysis, three cross sectional images at the distal, middle, and proximal of the diaphyseal VOI will be exported for analysis. For each image, the cortical bone area and 2nd moment of the area, as well as the Medial-Lateral and Anterior-Posterior diameters, will be calculated in a separate image processing program (IMAQ vision builder, Labview).

For the fibula osteotomy model, the same procedures will be performed as described above except the focus will be on the fracture site and surrounding bony regions. An additional region halfway between the fracture site and the knee will also be analyzed for comparison purposes.

### Mechanical Testing

After micro-CT scanning, the femurs will be mechanically tested via 4-point bending [42]. An electroforce system (ELF3200, Bose Corp., Eden Prairie, Minn.) with a custom testing apparatus will be used to stress the femurs to failure at a constant displacement rate of 0.05 mm/s. The femurs will be tested in the posterior to anterior direction so that the anterior side will be in tension. A 10-lb load cell (Sensotec, Columbus, Ohio, USA) will be used to measure the load applied to the bone, and the mid-diaphyseal displacement will be measured with a linear variable differential transducer. Load and displacement data will be acquired using the WinTest system (version 2.0; EnduraTec). The resulting load-displacement curves will be used to determine stiffness, yield load and displacement, ultimate load and displacement, post yield displacement for each femur. The linear region of the load-displacement curve in the first 0.1 mm of deflection will be determined; and stiffness will be measured as the slope of this linear region. The yield point will be defined as the point at which the secant stiffness reduced by 10% from the initial tangential stiffness. Failure will be defined as a sudden drop in load of over 10%. Ultimate load will be the maximum load attained before failure, and ultimate displacement will be the corresponding displacement. Post yield displacement will be calculated as the displacement at failure minus the displacement at the yield point. A displacement ratio will be calculated as the ratio of ultimate displacement to yield displacement to characterize the relative magnitudes of elastic and plastic deformation. Using these data and area data calculated from the mid-diaphysis CT imaging, the elastic modulus, yield stress, and ultimate stress will be determined.

### Example IV

### Testing the Effect of Periostin and Periostin Fragments on the in vitro Differentiation of Chondrogenic and Osteogenic Cells

### Cells and Cell Lines

All assays will utilize the following primary cells and cell lines that have previously been demonstrated to be important model systems for studying osteogenesis and chondrogenesis in vitro: (i) the C3H10T1/2 cell line as a model of chondrogenesis⁴⁸, (ii) the ST2 cell line as a model of cells that can differentiate into both chondrogenic and osteogenic lineage⁴⁸, (iii) primary culture of bone marrow stromal cells (BMSC), which can differentiate into osteoblasts⁴⁸, and (iv) primary mouse calvarial osteoblasts, which further differentiate in vitro^{49,50}. Each of the two primary culture systems will be derived from both periostin +/+ and −/− mice and they will be used as a model of non transformed bone progenitor cells⁵¹. All of these cell models will be evaluated using in vitro assays that assess cell adhesion, differentiation, proliferation, migration and invasion, which are all important cell behaviors typical of bone regeneration after fracture. Periostin will be modulated in a variety of ways: (i) adding purified protein, (ii) adding viruses (e.g., adenoviruses) that express periostin, and (iii) adding lentiviruses that express shRNA directed against periostin.

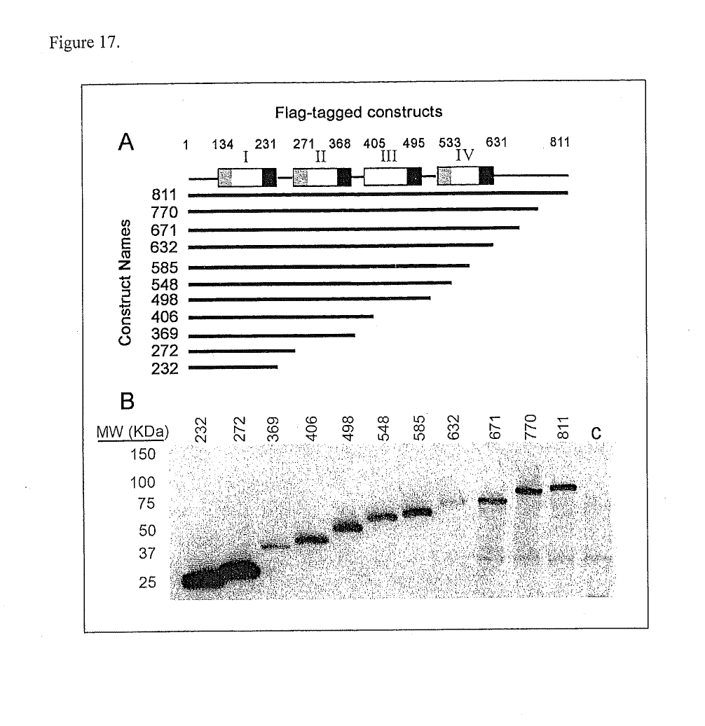

An extra feature of the in vitro assays is to use them to define the biologically active region of the periostin protein necessary for bone regeneration. Different series of periostin truncations are produced; one set that is FLAG-tagged, another set that is HIS6X tagged and another set that is hemagglutinin (HA)-tagged. Each truncation is encoded by a plasmid that can be expressed in eukaryotic cells (FIG. 17). The tag facilitates identification and distinction of the truncations from endogenous periostin and they also allow for purification from cell culture media. Therefore altered periostin-encoding nucleic acid can be transfected and/or transduced into cells or the purified truncated protein (e.g., fragment) can be added to cells, whichever is most advantageous for each assay.

Testing the Effect of Periostin and Periostin Truncations on the in vitro Differentiation of Chondrogenic and Osteogenic Cells.

The four cell culture models described above will be used in the following manner to ascertain the impact of periostin and periostin fragments on differentiation into chondroblasts or osteoblasts.

Periostin will be modulated in the C3H10T1/2 cultures by the following three approaches. (1) Periostin protein will be added at varying concentrations (0.1, 1.0, and 10 μg/ml) to establish the optimum concentration that promotes differentiation, with the control cultures not receiving any periostin protein. (2) Adenoviruses will be added that express full length periostin, with control cultures receiving an adenovirus that expresses green fluorescent protein (GFP). (3) Lentiviruses will be added that express shRNA which effectively abolishes periostin translation, with control cultures receiving lentiviruses that express shRNA directed against periostin but which are not effective in blocking expression.

C3H10T1/2 cells will be cultured for expansion without BMP-2, but to test differentiation they will be exposed to varying levels of periostin in the presence or absence of 300 ng/ml of BMP-2 (R&D Systems) while growing on collagen I coated dishes in DMEM supplemented with 10% FBS, 50 μg/ml ascorbic acid, and 5 mM β-glycerophosphate. The density of the cells will also be varied, grown either as a monolayer or in a micromass setting. The cells will be assayed for differentiation into chondroblasts by (i) Alcian blue staining at day 6, digital imaging and subsequent spectrophotometric reading⁵² and (ii) by quantitative (qRT-PCR) for markers of cartilage differentiation (Sox9 and Col2α1)⁴⁸ and normalization controls (18S and GAPDH)^{48, 53} on days 1,3, and 6.

To test differentiation in ST2 cells, periostin will be used according to the same methods as described above for the C3H10T1/2 cells. The ST2 cells are cultured for differentiation in RPMI-1640 with 10% FBS plus 300 ng/ml BMP-2 supplemented with 50 μg/ml ascorbic acid and 5 mM β-glycerophosphate while cultured on collagen I coated dishes. Chondrogenic differentiation of ST2 cells will be assayed as described for the C3H10T1/2 cells using Alcian blue, and qRT-PCR analysis of Sox9 and Col2α1 normalization controls (18S and GAPDH)^{48, 53}. The ST2 cells will also be assayed for osteoblast differentiation by (i) von Kossa staining on day 6^{54,55}, (ii) alkaline phosphate (ALP) staining on day 6^{54,55}, (iii) and qRT-PCR expression analysis evaluating the following bone differentiation markers⁴⁸: Runx2, bone sialoprotein (BSP), and osteocalcin (OC) on days 1, 3, and 6. The qRT-PCR will use 18S and GAPDH primers as standards for normalization as described^{48, 53}.

Bone marrow stromal cell (BMSC) primary cultures will be isolated by the method of Shirakawa⁵⁶. These cells differentiate into osteoblasts in DMEM plus 10% FBS and 300 ng/ml BMP-2 supplemented with 50 μg/ml ascorbic acid and 5 mM β-glycerophosphate on collagen I coated dishes. These cultures will not be used beyond five passages. These BMSC cells will be isolated from adult bone marrow of periostin +/+ and periostin −/− mice. The BMSC cultures will be assayed for osteoblast differentiation as described above for the ST2 cells (i.e., by ALP and Von Kossa staining on day 6 as well as qRT-PCR of Runx2, BSP and OC on days 1, 3, and 6 with normalization controls 18S and GAPDH^{48, 53}). If the periostin status of these cultures correlates with altered differentiation, then the periostin levels will be further modulated in the BMSC cell cultures by adding purified periostin protein or adenoviruses expressing periostin to the −/− cultures, as well as adding lentiviruses expressing shRNA to the +/+ cultures. These controls will ensure that the effect is due to periostin expression status and not a difference in isolation or culturing.

Calvarial cell primary cultures will be isolated from neonatal (˜24 hours old) periostin +/+ and periostin −/− mice as described⁵⁰. Because this culture requires ˜10 Day 1 neonates of the same genotype to be pooled to obtain enough cells, periostin matings of −/− males and females will be performed. This is possible since the periostin −/− mice are viable and fertile with the advantage that all of the offspring will be −/−. Matings of WT mice will generate the +/+ genotype. The calvarial cell cultures will not be used beyond five passages and for the differentiation experiments, they will be cultured in DMEM with 10% FBS supplemented with 10 mg/ml ascorbic acid and 500 mM β-glycerophosphate on collagen I coated dishes. The calvarial osteoblasts will be assayed for osteoblast differentiation by staining (ALP and Von Kossa) on day 6 as well as qRT-PCR of Runx2, BSP and OC on days 1, 3, and 6⁴⁸ with normalization controls (18S and GAPDH)^{48, 53}. If the periostin status of these cultures correlates with altered differentiation, then the periostin levels in the BMSC cell cultures will be further modulated by adding purified periostin protein or adenoviruses expressing periostin to the −/− cultures, as well as adding lentiviruses expressing shRNA to the +/+ cultures. These controls will ensure that the effect is due to periostin expression status and not a difference in isolation or culturing.

Primary cell lines will be isolated three separate times to allow for the variability of the isolation procedure. Cells will be plated in triplicate for each assay condition. To evaluate and quantify chondrocyte differentiation, digital images will be captured of Alcian blue staining. Morphometric analysis of Alcian blue-stained cultures will be used to determine the relative positive number of pixels of blue staining compared to total cell area, to assess the extent of differentiation. Alcian blue will also be extracted⁵² and the resulting data will be evaluated by student t-test.

Testing for the Ability of Bone Related Cells to Migrate on, and/or Invade into, Matrices Containing Periostin or Periostin Fragments

The four cell culture models described above will be evaluated in both 2-D and 3-D migrations assays. The 2-D or “scratch” assay will be performed by plating the cells at near confluency 24 hours prior to the “scratch” in medium with 1.5% serum. The “scratch” is performed with a 200 μl pipette tip, the cells are washed gently with PBS, and then medium is added to the cells, but the new medium does not contain serum. The serum is minimized in the setup and after the scratch to minimize its proliferative effects on cells, which could confound the determination of migration. The proliferation is assessed by using a 4 hour pulse of 10 uM BrdU and immunolabeling⁵⁷ with a FITC conjugated anti-BrdU antibody (Abcam) or immunostaining for PCNA⁵⁸. The minimum amount of serum may need to be determined for each cell type so that they remain viable but not proliferative. The “scratch” is marked at three discrete locations so that it can be digitally captured at exactly the same position over the time points of 0, 3, 6, 12, 24, and 48 hours after scratching the confluent monolayer. The surface area of the scratch on the digital images is measured using a Photoshop Adobe Creative Suite 4 program. Three different fields of the same scratch are analyzed per well of a 12 well dish and three wells are used per condition, with the results being averaged together and statistically analyzed using the student t-test and Anova. The migration of cells on various matrices (e.g., collagen, periostin, collagen+periostin, collagen+periostin truncations) can be assessed relatively quickly with this assay.

The 3-D migration and invasion assay will be performed by aggregating cells using the hanging drop method as described herein. The following day the clump of cells in the hanging drop is placed on top of a collagen gel (1.5 mg/ml) and incubated for 72 hours, fixed in 4% paraformaldehyde and immunostained or chemically stained prior to digital capture and morphometric analysis. First, the number of cells that have migrated out from the aggregate and the distance migrated on the top of the gel are calculated. Second, the number of cells that invade into the gel and the depth of invasion are quantified. At least ten aggregates will be scored, averaged, and statistically analyzed using the student t-test and Anova.

The 2-D and 3-D approaches described above will utilize full-length periostin or periostin truncations (e.g., fragments). The truncations will be compared to the full length periostin to determine which region contains the bioactivity of full-length periostin. Two series of truncations of periostin (10 different sizes) have been generated, one set is FLAG tagged and another set is hemagglutinin (HA) tagged. Each of these can be expressed in eukaryotic cells and then purified by affinity chromatography using the epitope tag (FIG. 17). Equimolar levels of the affinity purified truncations will be used in these assays.

### Testing for the Ability of Bone Related Cells to Adhere to Matrices Containing Periostin or Periostin Fragments.

The analysis of the four models of bone-related cells will be evaluated for adhesion to periostin matrices as described⁵⁹. Adhesion of the cells to various matrices such as collagen, periostin, periostin truncations (i.e., fragments) and/or combinations thereof, will be investigated. After the adhesion assays are completed, the numbers for each triplicate will be averaged, and the adhesion versus concentration plotted to assess the dependence of cell binding to substrate concentration in each assay. For each cell type, relative adhesive ability of the substrates will be compared to each other. As a control experiment, periostin truncations that are identified as having putative binding activity will be verified by a blocking experiment (i.e., pre-incubating the cells with the candidate truncation that was shown to have cell binding activity, prior to plating on a full-length periostin substrate). This will confirm specificity of the interaction.

Testing for the Proliferation of Bone Related Cells in Response to Treatment with Periostin or Periostin Fragments.

The analysis of the four models of bone related cells will be evaluated by both BrdU uptake and subsequent immunostaining as well as MTT assay, to assess proliferation rates as described⁵². The specific concentrations of periostin (or molar equivalents of periostin purified truncations) will be between 0.1, 1, and 10 μg/ml for this assay. Each assay will be done in triplicate for each cell culture model. In addition to adding exogenous periostin protein or purified truncations, these cells will be treated with adenoviruses that express periostin or periostin peptides or fragments and lentiviruses that decrease periostin expression via shRNA. In this way periostin and periostin expression can be modulated in the cell cultures in a variety of ways and the specific impact of various periostin levels on cell proliferation can be assessed in each cell type. This is particularly true of the primary culture from periostin −/− mice. If a distinct difference in comparing them to the cells from periostin +/+ mice calvarial osteoblasts is identified, the phenotype will be attempted to be rescued by adding back exogenous periostin to the periostin −/− cells. The converse will also be done (i.e., adding lentiviruses that express shRNA against periostin to decrease expression of the periostin +/+ cells) to see if there are changes in proliferation as compared to that observed for the periostin −/− cells. These controls will ensure that the effect is due to periostin expression status and not a difference in isolation or culturing.

If the data from these studies demonstrate that periostin is also expressed in the inflammatory period of fracture healing, cell culture models of inflammatory cells will be examined. The most likely cell types that would be added are primary fibroblasts⁶⁰ and primary neutrophils⁶¹ from the periostin −/− and +/+ genotypes. These would be utilized in a similar manner to the other primary cultures in testing proliferation, adhesion and migration.

The truncation analysis described herein will be used as a screen to identify which truncations to examine in vivo as described herein. In some experiments, the full length purified periostin protein will be compared to truncation #498 (FIG. 17), which has three out of four FAS domains with the most carboxyl one removed. This will allow for selection of amino or carboxyl domains of the protein that are important. If this truncation has the same bioactivity as the full length periostin in the in vitro assays, the 4 smaller truncations will be examined. If this truncation has altered bioactivity compared to the full length periostin then the 5 larger truncations will be examined. If we see an effect then we can map it to the specific location using additional truncations from the opposite direction and/or specific polypeptides spanning the region identified. This in vitro refinement of the smallest region of periostin that is biologically active and how it signals cells to change behavior will be initiated in these studies.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

### REFERENCES

1. Browner, B. Skeletal trauma basic science, management, and reconstruction (Saunders, Philadelphia, 2003).
2. Vaibhav, B., Nilesh, P., Vikram, S. & Anshul, C. Bone morphogenic protein and its application in trauma cases: a current concept update. Injury 38, 1227-35 (2007).
3. Lindner, V., Wang, Q., Conley, B. A., Friesel, R. E. & Vary, C. P. Vascular injury induces expression of periostin: implications for vascular cell differentiation and migration. Arterioscler Thromb Vasc Biol 25, 77-83 (2005).
4. Norris, R. A. et al. Periostin regulates collagen fibrillogenesis and the biomechanical properties of connective tissues. J Cell Biochem 101, 695-711 (2007).
5. Butcher, J. T., Norris, R. A., Hoffman, S., Mjaatvedt, C. H. & Markwald, R. R. Periostin promotes atrioventricular mesenchyme matrix invasion and remodeling mediated by integrin signaling through Rho/PI 3-kinase. Dev Biol 302, 256-66 (2007).
6. Gillan, L. et al. Periostin secreted by epithelial ovarian carcinoma is a ligand for alpha(V)beta(3) and alpha(V)beta(5) integrins and promotes cell motility. Cancer Res 62, 5358-64 (2002).
7. Takayama, G. et al. Periostin: a novel component of subepithelial fibrosis of bronchial asthma downstream of IL-4 and IL-13 signals. J Allergy Clin Immunol 118, 98-104 (2006).
8. Litvin, J. et al. Expression and function of periostin-isoforms in bone. J Cell Biochem 92, 1044-61 (2004).
9. Zhu, S. et al. Immunolocalization of Periostin-Like-Factor and Periostin During Embryogenesis. J Histochem Cytochem (2007).
10. Boyd H B, Lipinski S W & J H., W. Observations on non-union of the shafts of the long bones, with a statistical analysis of 842 patients. J Bone Joint Surg Am 43, 159-168 (1961).
11. Canale, S., Campbell, W. & LLC, M. C. Campbell's operative orthopaedics (MD Consult LLC, St. Louis, 2003).
12. AAOS. Future directions in musculoskeletal research; A summary report of the AAOS research committee panel studies. In. (2003).
13. AAOS. Research priorities for the unified musculoskeletal research agenda. In. (2005).
14. Lane, J. M. Bone morphogenic protein science and studies. J Orthop Trauma 19, S17-22 (2005).
15. Horiuchi, K. et al. Identification and characterization of a novel protein, periostin, with restricted expression to periosteum and periodontal ligament and increased expression by transforming growth factor beta. J Bone Miner Res 14, 1239-49 (1999).
16. Wilde, J., Yokozeki, M., Terai, K., Kudo, A. & Moriyama, K. The divergent expression of periostin mRNA in the periodontal ligament during experimental tooth movement. Cell Tissue Res 312, 345-51 (2003).
17. Oshima, A. et al. A novel mechanism for the regulation of osteoblast differentiation: transcription of periostin, a member of the fasciclin I family, is regulated by the bHLH transcription factor, twist. J Cell Biochem 86, 792-804 (2002).
18. Wu, X., W. Shi, and X. Cao, Multiplicity of BMP signaling in skeletal development. Ann N Y Acad Sci, 2007. 1116: p. 29-49.
19. Lane, J. M., Bone morphogenic protein science and studies. J Orthop Trauma, 2005. 19(10 Supply: p. S17-22.
20. Khosla, S., J. J. Westendorf, and M. J. Oursler, Building bone to reverse osteoporosis and repair fractures. J Clin Invest, 2008. 118(2): p. 421-8.
21. Inai, K., R. A. Norris, S. Hoffman, R. R. Markwald, and Y. Sugi, BMP-2 induces cell migration and periostin expression during atrioventricular valvulogenesis. Dev Biol, 2008. 315(2): p. 383-96.
22. Hadjiargyrou, M., F. Lombardo, S. Zhao, W. Ahrens, J. Joo, H. Ahn, M. Jurman, D. W. White, and C. T. Rubin, Transcriptional profiling of bone regeneration. Insight into the molecular complexity of wound repair. J Biol Chem, 2002. 277(33): p. 30177-82.
23. Norris, R. A., R. A. Moreno-Rodriguez, Y. Sugi, S. Hoffman, J. Amos, M. M. Hart, J. D. Potts, R. L. Goodwin, and R. R. Markwald, Periostin regulates atrioventricular valve maturation. Dev Biol, 2008. 316(2): p. 200-13.
24. Norris, R. A., B. Damon, V. Mironov, V. Kasyanov, A. Ramamurthi, R. Moreno-Rodriguez, T. Trusk, J. D. Potts, R. L. Goodwin, J. Davis, S. Hoffman, X. Wen, Y. Sugi, C. B. Kern, C. H. Mjaatvedt, D. K. Turner, T. Oka, S. J. Conway, J. D. Molkentin, G. Forgacs, and R. R. Markwald, Periostin regulates collagen fibrillogenesis and the biomechanical properties of connective tissues. J Cell Biochem, 2007. 101(3): p. 695-711.
25. Kii, I. and A. Kudo, [Periostin function in the periodontal ligament and the periosteum]. Clin Calcium, 2007. 17(2): p. 202-8.
26. Zhu, S., M. F. Barbe, N. Amin, S. Rani, S. N. Popoff, F. F. Safadi, and J. Litvin, Immunolocalization of Periostin-like factor and Periostin during embryogenesis. J Histochem Cytochem, 2008. 56(4): p. 329-45.
27. Horiuchi, K., N. Amizuka, S. Takeshita, H. Takamatsu, M. Katsuura, H. Ozawa, Y. Toyama, L. F. Bonewald, and A. Kudo, Identification and characterization of a novel protein, periostin, with restricted expression to periosteum and periodontal ligament and increased expression by transforming growth factor beta. J Bone Miner Res, 1999. 14(7): p. 1239-49.
28. Litvin, J., A. H. Selim, M. O. Montgomery, K. Lehmann, M. C. Rico, H. Devlin, D. P. Bednarik, and F. F. Safadi, Expression and function of periostin-isoforms in bone. J Cell Biochem, 2004. 92(5): p. 1044-61.
29. Bornstein, P. and E. H. Sage, Matricellular proteins: extracellular modulators of cell function. Curr Opin Cell Biol, 2002. 14(5): p. 608-16.
30. Butcher, J. T., R. A. Norris, S. Hoffman, C. H. Mjaatvedt, and R. R. Markwald, Periostin promotes atrioventricular mesenchyme matrix invasion and remodeling mediated by integrin signaling through Rho/PI 3-kinase. Dev Biol, 2007. 302(1): p. 256-66.
31. Oka, T., J. Xu, R. A. Kaiser, J. Melendez, M. Hambleton, M. A. Sargent, A. Lorts, E. W. Brunskill, G. W. Dorn, 2nd, S. J. Conway, B. J. Aronow, J. Robbins, and J. D. Molkentin, Genetic manipulation of periostin expression reveals a role in cardiac hypertrophy and ventricular remodeling. Circ Res, 2007. 101(3): p. 313-21.
32. Norris, R. A., T. K. Borg, J. T. Butcher, T. A. Baudino, I. Banerjee, and R. R. Markwald, Neonatal and adult cardiovascular pathophysiological remodeling and repair: developmental role of periostin. Ann N Y Acad Sci, 2008. 1123: p. 30-40.
33. Shimazaki, M., K. Nakamura, I. Kii, T. Kashima, N. Amizuka, M. Li, M. Saito, K. Fukuda, T. Nishiyama, S. Kitajima, Y. Saga, M. Fukayama, M. Sata, and A. Kudo, Periostin is essential for cardiac healing after acute myocardial infarction. J Exp Med, 2008. 205(2): p. 295-303.
34. Snider, P., R. B. Hinton, R. A. Moreno-Rodriguez, J. Wang, R. Rogers, A. Lindsley, F. Li, D. A. Ingram, D. Menick, L. Field, A. B. Firulli, J. D. Molkentin, R. Markwald, and S. J. Conway, Periostin is required for maturation and extracellular matrix stabilization of noncardiomyocyte lineages of the heart. Circ Res, 2008. 102(7): p. 752-60.
35. Shimazaki, M. and A. Kudo, Impaired capsule formation of tumors in periostin-null mice Biochem Biophys Res Commun, 2008. 367(4): p. 736-42.
36. Liang, C. C., A. Y. Park, and J. L. Guan, In vitro scratch assay: a convenient and inexpensive method for analysis of cell migration in vitro. Nat Protoc, 2007. 2(2): p. 329-33.
37. Chesterman, E. S., G. D. Gainey, A. C. Varn, R. E. Peterson, Jr., and M. J. Kern, Investigation of Prx1 protein expression provides evidence for conservation of cardiac-specific posttranscriptional regulation in vertebrates. Dev Dyn, 2001. 222(3): p. 459-70.
38. Kern, C. B., S. Hoffman, R. Moreno, B. J. Damon, R. A. Norris, E. L. Krug, R. R. Markwald, and C. H. Mjaatvedt, Immunolocalization of chick periostin protein in the developing heart. Anat Rec A Discov Mol Cell Evol Biol, 2005. 284(1): p. 415-23.
39. Kern, C. B., R. A. Norris, R. P. Thompson, W. S. Argraves, S. E. Fairey, L. Reyes, S. Hoffman, R. R. Markwald, and C. H. Mjaatvedt, Versican proteolysis mediates myocardial regression during outflow tract development. Dev Dyn, 2007.
40. Kern, C. B., W. O. Twal, C. H. Mjaatvedt, S. E. Fairey, B. P. Toole, M. L. Iruela-Arispe, and W. S. Argraves, Proteolytic cleavage of versican during cardiac cushion morphogenesis. Dev Dyn, 2006. 235(8): p. 2238-47.
41. Boyan, B. D., K. L. Wong, L. Wang, H. Yao, R. E. Guldberg, M. Drab, H. Jo, and Z. Schwartz, Regulation of growth plate chondrocytes by 1,25-dihydroxyvitamin D3 requires caveolae and caveolin-1. J Bone Miner Res, 2006. 21(10): p. 1637-47.
42. Rubin, J., Z. Schwartz, B. D. Boyan, X. Fan, N. Case, B. Sen, M. Drab, D. Smith, M. Aleman, K. L. Wong, H. Yao, H. Jo, and T. S. Gross, Caveolin-1 knockout mice have increased bone size and stiffness. J Bone Miner Res, 2007. 22(9): p. 1408-18.
43. Caffrey, J. M. and M. C. Farach-Carson, Vitamin D3 metabolites modulate dihydropyridine-sensitive calcium currents in clonal rat osteosarcoma cells. J Biol Chem, 1989. 264(34): p. 20265-74.
44. Wang, D., K. Christensen, K. Chawla, G. Xiao, P. H. Krebsbach, and R. T. Franceschi, Isolation and characterization of MC3T3-E1 preosteoblast subclones with distinct in vitro and in vivo differentiation/mineralization potential. J Bone Miner Res, 1999. 14(6): p. 893-903.
45. Burger, E. H., P. M. Boonekamp, and P. J. Nijweide, Osteoblast and osteoclast precursors in primary cultures of calvarial bone cells. Anat Rec, 1986. 214(1): p. 32-40.
46. Giaretti, W., M. Nusse, S. Bruno, A. Di Vinci, and E. Geido, A new method to discriminate G1, S, G2, M, and G1 postmitotic cells. Exp Cell Res, 1989. 182(1): p. 290-5.
47. Kohler, T., F. Prols, and B. Brand-Saberi, PCNA in situ hybridization: a novel and reliable tool for detection of dynamic changes in proliferative activity. Histochem Cell Biol, 2005. 123(3): p. 315-27.
48. Tominaga, H., S. Maeda, H. Miyoshi, K. Miyazono, S. Komiya, and T. Imamura, Expression of osterix inhibits bone morphogenetic protein-induced chondrogenic differentiation of mesenchymal progenitor cells. J Bone Miner Metab, 2009. 27(1): p. 36-45.
49. Connerney, J., V. Andreeva, Y. Leshem, C. Muentener, M. A. Mercado, and D. B. Spicer, Twist1 dimer selection regulates cranial suture patterning and fusion. Dev Dyn, 2006. 235(5): p. 1345-57.
50. Clemens, T. L., H. Tang, S. Maeda, R. A. Kesterson, F. Demayo, J. W. Pike, and C. M. Gundberg, Analysis of osteocalcin expression in transgenic mice reveals a species difference in vitamin D regulation of mouse and human osteocalcin genes. J Bone Miner Res, 1997. 12(10): p. 1570-6.
51. Burger, E. H., P. M. Boonekamp, and P. J. Nijweide, Osteoblast and osteoclast precursors in primary cultures of calvarial bone cells. Anat Rec, 1986. 214(1): p. 32-40.
52. Peterson, R. E., S. Hoffman, and M. J. Kern, Opposing roles of two isoforms of the Prx1 homeobox gene in chondrogenesis. Dev Dyn, 2005. 233(3): p. 811-21.
53. Kellum, E., H. Starr, P. Arounleut, D. Immel, S. Fulzele, K. Wenger, and M. W. Hamrick, Myostatin (GDF-8) deficiency increases fracture callus size, Sox-5 expression, and callus bone volume. Bone, 2009. 44(1): p. 17-23.
54. Hayashi, M., S. Maeda, H. Aburatani, K. Kitamura, H. Miyoshi, K. Miyazono, and T. Imamura, Pitx2 prevents osteoblastic transdifferentiation of myoblasts by bone morphogenetic proteins. J Biol Chem, 2008. 283(1): p. 565-71.
55. Maeda, S., M. Hayashi, S. Komiya, T. Imamura, and K. Miyazono, Endogenous TGF-beta signaling suppresses maturation of osteoblastic mesenchymal cells. EMBO J, 2004. 23(3): p. 552-63.
56. Shirakawa, K., S. Maeda, T. Gotoh, M. Hayashi, K. Shinomiya, S. Ehata, R. Nishimura, M. Mori, K. Onozaki, H. Hayashi, S. Uematsu, S. Akira, E. Ogata, K. Miyazono, and T. Imamura, CCAAT/enhancer-binding protein homologous protein (CHOP) regulates osteoblast differentiation. Mol Cell Biol, 2006. 26(16): p. 6105-16.
57. Giaretti, W., M. Nusse, S. Bruno, A. Di Vinci, and E. Geido, A new method to discriminate G1, S, G2, M, and G1 postmitotic cells. Exp Cell Res, 1989. 182(1): p. 290-5.
58. Kohler, T., F. Prols, and B. Brand-Saberi, PCNA in situ hybridization: a novel and reliable tool for detection of dynamic changes in proliferative activity. Histochem Cell Biol, 2005. 123(3): p. 315-27.
59. Butcher, J. T., R. A. Norris, S. Hoffman, C. H. Mjaatvedt, and R. R. Markwald, Periostin promotes atrioventricular mesenchyme matrix invasion and remodeling mediated by integrin signaling through Rho/PI 3-kinase. Dev Biol, 2007. 302(1): p. 256-66.
60. Conner, D. A., Mouse embryo fibroblast (MEF) feeder cell preparation. Curr Protoc Mol Biol, 2001. Chapter 23: p. Unit 23 2.
61. Luo, Y. and M. E. Dorf, Isolation of mouse neutrophils. Curr Protoc Immunol, 2001. Chapter 3: p. Unit 3 20.

TABLE 1
Murine periostin peptides

| | | |
|---|---|---|
| 1: | INPANANSYYDKVLAHSRIR | (SEQ ID NO: 1) |
| 2: | HSRIRGRDQGPNVCALQQIL | (SEQ ID NO: 2) |
| 3: | LQQILGTKKKYFSSCKNWYQ | (SEQ ID NO: 3) |
| 4: | KNWYQGAICGKKTTVLYECC | (SEQ ID NO: 4) |
| 5: | LYECCPGYMRMEGMKGCPAV | (SEQ ID NO: 5) |
| 6: | GCPAVMPIDHVYGTLGIVGA | (SEQ ID NO: 6) |
| 7: | GIVGATTTQHYSDVSKLREE | (SEQ ID NO: 7) |
| 8: | KLREEIEGKGSYTYFAPSNE | (SEQ ID NO: 8) |
| 9: | APSNEAWENLDSDIRRGLEN | (SEQ ID NO: 9) |
| 10: | RGLENNVNVELLNALHSHMV | (SEQ ID NO: 10) |
| 11: | HSHMVNKRMLTKDLKHGMVI | (SEQ ID NO: 11) |
| 12: | HGMVIPSMYNNLGLFINHYP | (SEQ ID NO: 12) |
| 13: | INHYPNGVVTVNCARVIHGN | (SEQ ID NO: 13) |
| 14: | VIHGNQIATNGVVHVIDRVL | (SEQ ID NO: 14) |
| 15: | IDRVLTQIGTSIQDFLEAED | (SEQ ID NO: 15) |
| 16: | LEAEDDLSSFRAAAITSDLL | (SEQ ID NO: 16) |
| 17: | TSDLLESLGRDGHFTLFAPT | (SEQ ID NO: 17) |
| 18: | LFAPTNEAFEKLPRGVLERI | (SEQ ID NO: 18) |
| 19: | VLERIMGDKVASEALMKYHI | (SEQ ID NO: 19) |
| 20: | MKYHILNTLQCSEAITGGAV | (SEQ ID NO: 20) |
| 21: | TGGAVFETMEGNTIEIGCEG | (SEQ ID NO: 21) |
| 22: | IGCEGDSISINGIKMVNKKD | (SEQ ID NO: 22) |
| 23: | VNKKDIVTKNGVIHLIDEVL | (SEQ ID NO: 23) |
| 24: | IDEVLIPDSAKQVIELAGKQ | (SEQ ID NO: 24) |
| 25: | LAGKQQTTFTDLVAQLGLAS | (SEQ ID NO: 25) |
| 26: | LGLASSLKPDGEYTLLAPVN | (SEQ ID NO: 26) |
| 27: | LAPVNNAFSDDTLSMDQRLL | (SEQ ID NO: 27) |
| 28: | DQRLLKLILQNHILKVKVGL | (SEQ ID NO: 28) |
| 29: | VKVGLSDLYNGQILETIGGK | (SEQ ID NO: 29) |
| 30: | TIGGKQLRVFVYRTAICIEN | (SEQ ID NO: 30) |
| 31: | ICIENSCMVRGSKQGRNGAI | (SEQ ID NO: 31) |
| 32: | RNGAIHIFREIIQPAEKSLH | (SEQ ID NO: 32) |
| 33: | EKSLHDKLRQDKRFSIFLSL | (SEQ ID NO: 33) |
| 34: | IFLSLLEAADLKDLLTQPGD | (SEQ ID NO: 34) |
| 35: | TQPGDWTLFAPTNDAFKGMT | (SEQ ID NO: 35) |
| 36: | FKGMTSEERELLIGDKNALQ | (SEQ ID NO: 36) |
| 37: | KNALQNIILYHLTPGVYIGK | (SEQ ID NO: 37) |
| 38: | VYIGKGFEPGVTNILKTTQG | (SEQ ID NO: 38) |
| 39: | KTTQGSKIYLKGVNETLLVN | (SEQ ID NO: 39) |
| 40: | TLLVNELKSKESDIMTTNGV | (SEQ ID NO: 40) |
| 41: | TTNGVIHVVDKLLYPADIPV | (SEQ ID NO: 41) |
| 42: | ADIPVGNDQLLELLNKLIKY | (SEQ ID NO: 42) |
| 43: | KLIKYIQIKFVRGSTFKEIP | (SEQ ID NO: 43) |
| 44: | FKEIPMTVYTTKIITKVVEP | (SEQ ID NO: 44) |
| 45: | KVVEPKIKVIQGSLQPIIKT | (SEQ ID NO: 45) |
| 46: | PIIKTEGPAMTKIQIEGDPD | (SEQ ID NO: 46) |
| 47: | EGDPDFRLIKEGETVTEVIH | (SEQ ID NO: 47) |
| 48: | TEVIHGEPVIKKYTKIIDGV | (SEQ ID NO: 48) |
| 49: | IIDGVPVEITEKQTREERII | (SEQ ID NO: 49) |
| 50: | EERIITGPEIKYTRISTGGG | (SEQ ID NO: 50) |
| 51: | STGGGETGETLQKFLQKEVS | (SEQ ID NO: 51) |
| 52: | QKEVSKVTKFIEGGDGHLFE | (SEQ ID NO: 52) |
| 53: | GHLFEDEEIKRLLQGDTPAK | (SEQ ID NO: 53) |
| 54: | DTPAKKIPANKRVQGPRRRS | (SEQ ID NO: 54) |
| 55: | IPANKRVQGPRRRSREGRSQ | (SEQ ID NO: 55) |

TABLE 2
Human periostin peptides

| | | |
|---|---|---|
| 1. | MIPFLPMFSLLLLLIVNPIN | (SEQ ID NO: 56) |
| 2. | VNPINANNHYDKILAHSRIR | (SEQ ID NO: 57) |
| 3. | HSRIRGRDQGPNVCALQQIL | (SEQ ID NO: 58) |
| 4. | LQQILGTKKKYFSTCKNWYK | (SEQ ID NO: 59) |
| 5. | KNWYKKSICGQKTTVLYECC | (SEQ ID NO: 60) |
| 6. | LYECCPGYMRMEGMKGCPAV | (SEQ ID NO: 61) |
| 7. | GCPAVLPIDHVYGTLGIVGA | (SEQ ID NO: 62) |
| 8. | GIVGATTTQRYSDASKLREE | (SEQ ID NO: 63) |
| 9. | KLREEIEGKGSFTYFAPSNE | (SEQ ID NO: 64) |
| 10. | APSNEAWDNLDSDIRRGLES | (SEQ ID NO: 65) |
| 11. | RGLESNVNVELLNALHSHMI | (SEQ ID NO: 66) |
| 12. | HSHMINKRMLTKDLKNGMII | (SEQ ID NO: 67) |
| 13. | NGMIIPSMYNNLGLFINHYP | (SEQ ID NO: 68) |
| 14. | INHYPNGVVTVNCARIIHGN | (SEQ ID NO: 69) |
| 15. | IIHGNQIATNGVVHVIDRVL | (SEQ ID NO: 70) |
| 16. | IDRVLTQIGTSIQDFIEAED | (SEQ ID NO: 71) |
| 17. | IEAEDDLSSFRAAAITSDIL | (SEQ ID NO: 72) |
| 18. | TSDILEALGRDGHFTLFAPT | (SEQ ID NO: 73) |
| 19. | LFAPTNEAFEKLPRGVLERI | (SEQ ID NO: 74) |
| 20. | VLERIMGDKVASEALMKYHI | (SEQ ID NO: 75) |
| 21. | MKYHILNTLQCSESIMGGAV | (SEQ ID NO: 76) |
| 22. | MGGAVFETLEGNTIEIGCDG | (SEQ ID NO: 77) |
| 23. | IGCDGDSITVNGIKMVNKKD | (SEQ ID NO: 78) |
| 24. | VNKKDIVTNNGVIHLIDQVL | (SEQ ID NO: 79) |
| 25. | IDQVLIPDSAKQVIELAGKQ | (SEQ ID NO: 80) |
| 26. | LAGKQQTTFTDLVAQLGLAS | (SEQ ID NO: 81) |
| 27. | LGLASALRPDGEYTLLAPVN | (SEQ ID NO: 82) |
| 28. | LAPVNNAFSDDTLSMDQRLL | (SEQ ID NO: 83) |
| 29. | DQRLLKLILQNHILKVKVGL | (SEQ ID NO: 84) |
| 30. | VKVGLNELYNGQILETIGGK | (SEQ ID NO: 85) |
| 31. | TIGGKQLRVFVYRTAVCIEN | (SEQ ID NO: 86) |
| 32. | VCIENSCMEKGSKQGRNGAI | (SEQ ID NO: 87) |
| 33. | RNGAIHIFREIIKPAEKSLH | (SEQ ID NO: 88) |
| 34. | EKSLHEKLKQDKRFSTFLSL | (SEQ ID NO: 89) |
| 35. | TFLSLLEAADLKELLTQPGD | (SEQ ID NO: 90) |
| 36. | TQPGDWTLFVPTNDAFKGMT | (SEQ ID NO: 91) |
| 37. | FKGMTSEEKEILIRDKNALQ | (SEQ ID NO: 92) |
| 38. | KNALQNIILYHLTPGVFIGK | (SEQ ID NO: 93) |
| 39. | VFIGKGFEPGVTNILKTTQG | (SEQ ID NO: 94) |
| 40. | KTTQGSKIFLKEVNDTLLVN | (SEQ ID NO: 95) |
| 41. | TLLVNELKSKESDIMTTNGV | (SEQ ID NO: 96) |
| 42. | TTNGVIHVVDKLLYPADTPV | (SEQ ID NO: 97) |
| 43. | ADTPVGNDQLLEILNKLIKY | (SEQ ID NO: 98) |
| 44. | KLIKYIQIKFVRGSTFKEIP | (SEQ ID NO: 99) |
| 45. | FKEIPVTVYTTKIITKVVEP | (SEQ ID NO: 100) |
| 46. | KVVEPKIKVIEGSLQPIIKT | (SEQ ID NO: 101) |
| 47. | PIIKTEGPTLTKVKIEGEPE | (SEQ ID NO: 102) |
| 48. | EGEPEFRLIKEGETITEVIH | (SEQ ID NO: 103) |
| 49. | TEVIHGEPIIKKYTKIIDGV | (SEQ ID NO: 104) |
| 50. | IIDGVPVEITEKETREERII | (SEQ ID NO: 105) |
| 51. | EERIITGPEIKYTRISTGGG | (SEQ ID NO: 106) |
| 52. | STGGGETEETLKKLLQEEVT | (SEQ ID NO: 107) |
| 53. | QEEVTKVTKFIEGGDGHLFE | (SEQ ID NO: 108) |
| 54. | GHLFEDEEIKRLLQGDTPVR | (SEQ ID NO: 109) |
| 55. | DTPVRKLQANKKVQGSRRRL | (SEQ ID NO: 110) |
| 56. | LQANKKVQGSRRRLREGRSQ | (SEQ ID NO: 111) |

What is claimed is:

1. A method of increasing bone production in a subject, comprising administering to the subject an effective amount of a periostin protein or a biologically active fragment thereof and/or a periostin peptide, thereby increasing bone production in the subject.

2. A method of decreasing healing time of a bone fracture in a subject in need thereof, comprising administering to the subject an effective amount of a periostin protein or a biologically active fragment thereof and/or a periostin peptide, thereby decreasing healing time of a bone fracture in the subject.

3. The method of claim 1, wherein the periostin protein or biologically active fragment thereof and/or peptide is administered directly to an injury site, wound site and/or surgical site in the subject.

4. The method of claim 2, wherein the periostin protein or biologically active fragment thereof and/or peptide is administered directly to an injury site, wound site and/or surgical site in the subject.

5. The method of claim 1, wherein the periostin protein or biologically active fragment thereof and/or peptide is administered to the subject intravenously, orally and/or transdermally.

6. The method of claim 2, wherein the periostin protein or biologically active fragment thereof and/or peptide is administered to the subject intravenously, orally and/or transdermally.

7. The method of claim 1, wherein the effective amount of the periostin protein or biologically active fragment thereof or the peptide is in the range of about 1 microgram/ml to about 500 milligrams/ml.

8. The method of claim 2, wherein the effective amount of the periostin protein or biologically active fragment thereof or the peptide is in the range of about 1 microgram/ml to about 500 milligrams/ml.

9. The method of claim 1, further comprising administering to the subject an agent selected from the group consisting of:
a) collagen;
b) a hydrogel;
c) a demineralized bone matrix;
d) an organic sponge;
e) an implantable matrix;
f) a bone chip; and
g) any combination of (a)-(f) above.

10. The method of claim 2, further comprising administering to the subject an agent selected from the group consisting of:
a) collagen;
b) a hydrogel;
c) a demineralized bone matrix;
d) an organic sponge;
e) an implantable matrix;
f) a bone chip; and
g) any combination of (a)-(f) above.

11. The method of claim 1, further comprising administering to the subject an agent selected from the group consisting of:
a) a differentiation stimulating agent;
b) a chemotaxis stimulating agent;
c) a proliferation stimulating agent;
d) a mobilization stimulating agent; and
e) any combination of (a)-(d) above.

12. The method of claim 2, further comprising administering to the subject an agent selected from the group consisting of:
a) a differentiation stimulating agent;
b) a chemotaxis stimulating agent;
c) a proliferation stimulating agent;
d) a mobilization stimulating agent; and
e) any combination of (a)-(d) above.

13. The method of claim 9, further comprising administering to the subject an agent selected from the group consisting of:
a) a differentiation stimulating agent;
b) a chemotaxis stimulating agent;
c) a proliferation stimulating agent;
d) a mobilization stimulating agent; and
e) any combination of (a)-(d) above.

14. The method of claim 10, further comprising administering to the subject an agent selected from the group consisting of:
a) a differentiation stimulating agent;
b) a chemotaxis stimulating agent;
c) a proliferation stimulating agent;
d) a mobilization stimulating agent; and
e) any combination of (a)-(d) above.
